(19) United States
(12) Patent Application Publication
(10) Pub. No.: US 2024/0122701 A1
(43) Pub. Date: Apr. 18, 2024

(54) Devices, Systems, and Methods for a Valve Replacement

(71) Applicant: ReValve Solutions Inc., Bainbridge Island, WA (US)

(72) Inventors: Julie Logan Sands, McLean, WA (US); Kenneth Eugene Perry, Bainbridge Island, WA (US); Anthony Zoltan Zador, Bainbridge Island, WA (US)

(73) Assignee: ReValve Solutions Inc., Bainbridge Island, WA (US)

(21) Appl. No.: 18/275,988

(22) PCT Filed: Feb. 4, 2022
(86) PCT No.: PCT/US22/15360

§ 371 Date: Aug. 4, 2023

Related U.S. Application Data
(63) Continuation of application No. PCT/US21/39451, filed on Jun. 28, 2021.
Application No. PCT/US21/39451 is a continuation of application No. PCT/US21/51828, filed on Sep. 23, 2021.
Application No. PCT/US21/51828 is a continuation of application No. PCT/US21/32817, filed on May 17, 2021.
Application No. PCT/US21/32817 is a continuation of application No. PCT/US21/38886, filed on Jun. 24, 2021.
(60) Provisional application No. 63/145,848, filed on Feb. 4, 2021.

(51) Int. Cl. A61F 2/24

(52) U.S. Cl. CPC A61F 2/2418; A61F 2220/0008; A61F 2250/0014; A61F 2250/006

(57) ABSTRACT

Disclosed is a valve replacement comprising a braided helical design that mimics the heart's natural movement. The braided wire frame of the disclosed valve replacement may be compressed to enable compact and secure delivery into the heart and convenient control during implantation as well as the expansion and retraction when implanted or removed/replaced, preferably entirely via a catheter. The valve replacement may comprise a one-piece system comprising an adapter body with engaging mechanisms that secure to the heart, and a valve assembly with leaflets positioned within the adapter body. The valve replacement may also comprise a two-piece system comprising an adapter body and valve assembly that are compatible with each other yet wherein the valve assembly may be removable from the adapter body such that both can be delivered together or separately and such that the adapter body may remain implanted while the valve assembly may be removed and replaced.

### CROSS-REFERENCE TO RELATED APPLICATIONS

This application claims priority and benefit to: U.S. Provisional Application No. 63/145,878, filed on Feb. 2, 2021, entitled “Devices, Systems, and Methods for a Self-Adapting Valve Attachment”; International Application No. PCT/US/21/39451, filed on Jun. 28, 2021, entitled “Devices, Systems, and Methods for a Heart-Valve Annulus Reinforcer”; International Application No. PCT/US21/51828, filed on Sep. 23, 2021, entitled “Devices, Systems, and Methods for an Implantable Heart-Valve Adapter”; International Application No. PCT/US21/32817, filed on May 17, 2021, entitled “Devices, Systems, and Methods for a Collapsible and Expandable Replacement Heart Valve”; and International Application No. PCT/US21/38886, filed on Jun. 24, 2021, entitled “Devices, Systems, and Methods for a Collapsible Replacement Heart Valve”—the contents all of which are incorporated herein by this reference as though set forth in their entirety.

### FIELD OF USE

The present disclosure relates generally to replacement heart-valve technology, and more specifically to devices, systems, and methods for a valve replacement comprising a one-piece system and a two-piece system. Characteristics of the discussed valve replacement comprise high flexibility, resiliency, conformality, and functionality as a replaceable heart valve.

### BACKGROUND

Heart-valve intervention, such as full open-heart surgery, is often required to treat diseases of one or more of the four heart valves (which work together to keep blood properly flowing through the heart). Replacement and/or repair of a heart valve is often required when a valve is “leaky” (e.g., there is valve regurgitation) or when a valve is narrowed and does not open properly (e.g., valve stenosis). Heart-valve replacement, such as mitral-valve or tricuspid-valve replacement, typically involves replacement of the heart's original (native) valve with a replacement mechanical and/or tissue (bioprosthetic) valve. Common problems with the replacement of valves and/or the frames carrying them include degradation of the leaflets (valve-like structure); breaking or failing frames, particularly with laser-cut nitinol frames; and undesirable changing in size of the native valve annulus. Replacement heart valves pose additional problems after they are implanted. For example, the replacement valve may move or migrate after it is placed in a desired location in the heart, or its location may not permit proper directional flow of blood through other parts of the organ, such as the outflow tract of the left ventricle. Replacement valves are also not readily retrievable, most often because such removal can damage the surrounding heart tissue. This can be particularly problematic, for example, if the replacement valve is not properly and accurately placed into position when it is implanted in the native heart, as well as when the replacement valve starts failing, which may occur soon or years after initial implantation. An additional problem is that typical replacement valves, especially laser-cut valve frames, are relatively stiff and inflexible, resulting in a valve that does not flex with the dynamic movements of the pumping heart. Such inflexible valves do not conform to such dynamic movements, which can cause trauma to the heart surfaces, cause breaks in the frame itself, otherwise cause or exacerbate problems during or after implantation.

Thus, what is needed are devices, systems, and methods for a valve replacement that enables compact and secure delivery into the heart and convenient control of both the valve replacement during implantation as well as the expansion and retraction of the valve replacement when being implanted or removed/replaced, preferably entirely via a catheter. Also needed are devices, systems, methods for ensuring proper directional flow of blood through the heart during and after a valve-replacement procedure.

Such devices, systems, and methods should provide the functionality of a one-piece system comprising both an adapter body with engaging mechanisms that secure to the heart and a valve assembly with leaflets that is positioned within the adapter body. Such devices, systems, and methods should also provide the functionality of a two-piece system comprising an adapter body and valve assembly that are compatible with each other yet wherein the valve assembly may be removable from the adapter body such that both can be delivered together or separately and such that the adapter body may remain implanted while the valve assembly may be removed and replaced.

### SUMMARY OF THE DISCLOSURE

The following presents a simplified overview of the example embodiments in order to provide a basic understanding of some embodiments of the present disclosure. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented herein below. It is to be understood that both the following general description and the following detailed description are exemplary and explanatory only and are not restrictive.

The present disclosure is directed to devices, systems, and method for a valve replacement that serves the purpose of anchoring, sealing, and controlling the position of the leaflets and sub-valvular structure. The replacement is highly flexible, resilient, fatigue resistant, and securable to the native valve tissue. And it is self-adapting, meaning, it adapts to—and in addition, also supports—the natural movement of the heart. In a preferred embodiment, the valve replacement comprises a collapsible adapter body that attaches to the native valve tissue and provides a sealing portion. The valve replacement comprises a frame optimized for effective sealing and fixation to the valve, wherein the design of the adapting frame is anatomically inspired and designed to maximize ventricular filling and minimize outflow tract obstruction. The valve replacement—whether as a one- or two-piece system—further comprises a valve assembly, wherein the valve assembly comprises leaflets and is compatible to reside within the adapting frame.

The present disclosure provides for a valve replacement that—due to its braided-wire frame design—is compressible to a smaller profile when compared to the prior art, wherein the smaller compressed profile allows for delivery via not only transapical approaches but also transfemoral and transseptal approaches. The two-piece system disclosed herein allows for a further lower profile because the adapting frame and the valve assembly may be delivered as two separate devices.

Still other advantages, embodiments, and features of the subject disclosure will become readily apparent to those of ordinary skill in the art from the following description wherein there is shown and described a preferred embodiment of the present disclosure, simply by way of illustration of one of the best modes best suited to carry out the subject disclosure. As will be realized, the present disclosure is capable of other different embodiments and its several details are capable of modifications in various obvious embodiments all without departing from, or limiting, the scope herein. Accordingly, the drawings and descriptions will be regarded as illustrative in nature and not as restrictive.

### BRIEF DESCRIPTION OF THE DRAWINGS

The accompanying drawings, which are incorporated in and constitute a part of this specification, illustrate embodiments of the disclosure and together with the general description of the disclosure given above and the detailed description of the drawings given below, serve to explain the principles of the disclosure. In certain instances, details that are not necessary for an understanding of the disclosure or that render other details difficult to perceive may have been omitted.

FIG. 1 generally illustrates an embodiment of a valve replacement as disclosed herein.

FIGS. 2A-2C generally illustrate an embodiment of a valve replacement as disclosed herein.

FIG. 3 generally illustrates an embodiment of a valve replacement as disclosed herein.

FIG. 4 generally illustrates an embodiment of a valve replacement as disclosed herein.

FIG. 5 generally illustrates an embodiment of a valve replacement as disclosed herein.

FIGS. 6A and 6B generally illustrate embodiments of a valve replacement as disclosed herein.

FIGS. 7A-7D generally illustrate embodiments of a valve replacement as disclosed herein.

FIG. 8A generally illustrates the helical functionality of the human heart.

FIG. 8B generally illustrate embodiments of a valve replacement as disclosed herein.

FIG. 9 generally illustrates an embodiment of a valve replacement as disclosed herein.

FIGS. 10A and 10B generally illustrate embodiments of a valve replacement as disclosed herein.

FIGS. 11A and 11B generally illustrate embodiments of a valve replacement as disclosed herein.

FIGS. 12A-12E generally illustrate embodiments of a valve replacement as disclosed herein.

FIGS. 13A-13D generally illustrate embodiments of a valve replacement as disclosed herein.

FIGS. 14A-14E generally illustrate embodiments of a valve replacement as disclosed herein.

FIGS. 15A and 15B generally illustrate embodiments of a valve replacement as disclosed herein.

FIGS. 16A and 16D generally illustrate embodiments of a valve replacement as disclosed herein.

FIGS. 17A-17D generally illustrate embodiments of a valve replacement as disclosed herein.

FIGS. 18A and 18B generally illustrate embodiments of a valve replacement as disclosed herein.

FIGS. 19A and 19B generally illustrate embodiments of a valve replacement as disclosed herein.

FIG. 20 generally illustrates an embodiment of a valve replacement as disclosed herein.

### DETAILED DESCRIPTION OF EMBODIMENTS

Before the present systems and methods are disclosed and described, it is to be understood that the systems and methods are not limited to specific methods, specific components, or to particular implementations. It is also to be understood that the terminology used herein is for the purpose of describing particular embodiments only and is not intended to be limiting. Various embodiments are described with reference to the drawings. In the following description, for purposes of explanation, numerous specific details are set forth in order to provide a thorough understanding of one or more embodiments. It may be evident, however, that the various embodiments may be practiced without these specific details. In other instances, well-known structures and devices are shown in block diagram form to facilitate describing these embodiments.

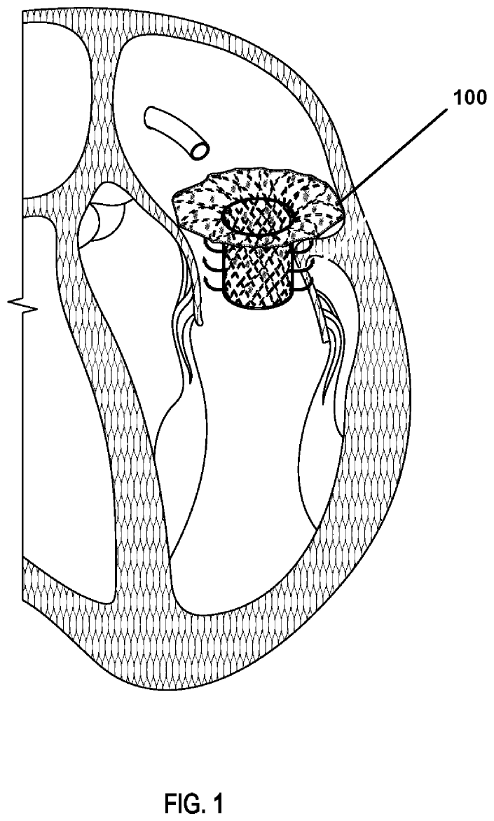

FIG. 1 generally illustrates an embodiment of a valve replacement as disclosed herein. FIG. 1 discloses a valve replacement (“Valve Replacement”) 100 implanted in a malfunctioning mitral valve. The Valve Replacement, however, is not limited to compatibility with only the mitral valve and may be also implanted in the tricuspid valve. In a preferred embodiment, the Valve Replacement comprises a braided, collapsible frame and a braided valve-and-leaflet assembly that together serve to provide a sealing portion.

As set forth herein, the compatibility of the collapsible frame and leaflet assembly may be performed in various embodiments. In one embodiment, the Valve Replacement may comprise the frame and valve assembly as a two-piece apparatus (referred to herein for ease of reference as the “Two-Piece System”). In another embodiment, the Valve Replacement may comprise the frame and valve assembly as a one-piece apparatus (referred to herein for ease of reference as the “One-Piece System”). Regardless of the embodiments, the Valve Replacement may further comprise attachments and additional features for catheter delivery, positioning, partial deployment, and retrieval.

Two-Piece Valve Replacement Overview

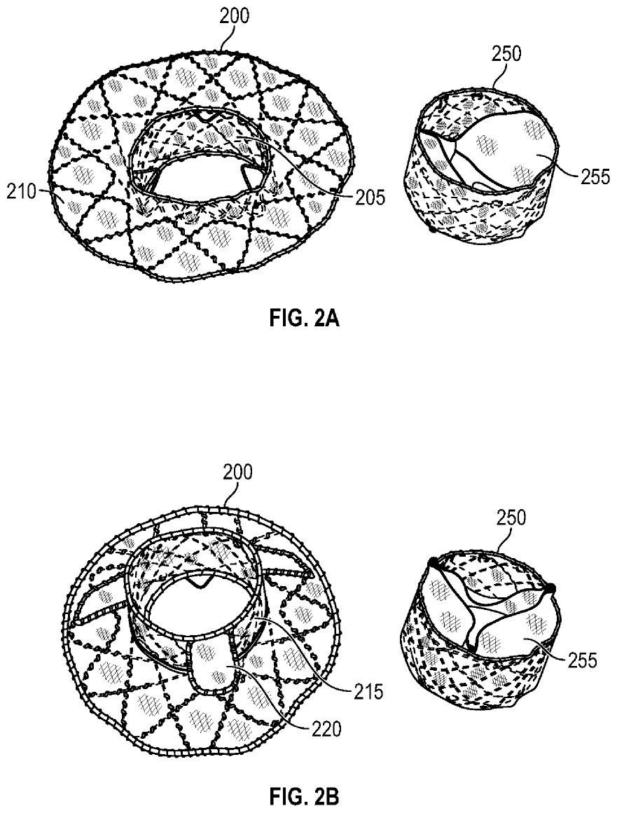

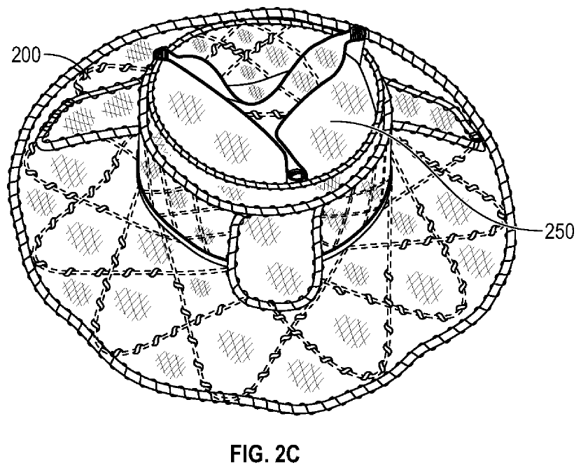

FIGS. 2A-2C generally illustrate an embodiment of a valve replacement as disclosed herein. FIGS. 2A-2C disclose embodiments of a Two-Piece System. As shown in FIG. 2A, the Two-Piece System comprises a heart-valve frame 200 (referred to herein for ease of reference as the “Adapter”) and a heart-valve assembly 250 (referred to herein for ease of reference as the “Valve Assembly”). In one embodiment, the Adapter 200 comprises an opening 205 that is compatible with the Valve Assembly 250. The Adapter 200 further comprises a sealing skirt 210 at the top, a body portion 215, and one or more anchors 220 extending out from the bottom of the body portion 215. In an embodiment, the Valve Assembly 250 comprises a leaflet-structure component 255 that enables blood flow through the Valve Assembly 250.

FIG. 2C discloses the Valve Replacement as a Two-Piece System wherein the Adapter 200 and the Valve Assembly 250 are cooperatively sized and configured together. The Adapter 200 and the Valve Assembly 250 may fit as a single unit and be compressed to be inserted into a heart catheter for delivery to a target valve, i.e., in an as-connected form where the two portions are mechanically linked together. This configuration advantageously allows the delivery and control of both portions of the Valve Replacement.

The Adapter 200 and the Valve Assembly 250 may also be carried in a delivery catheter in an unconnected form where the two portions are not mechanically linked together. This configuration advantageously allows the delivery catheter to independently control each of the portions and can also increase the flexibility and torsion characteristics of the delivery catheter containing the two portions, which can be advantageous both while conveying the delivery catheter to through the patient's body, the vasculature, the desired target, and while delivering the replacement valve at/to the target. In such embodiments, the Adapter 200 and the Valve Assembly 250, as separately delivered portions, may both be further compressed, enabling a low profile that is conducive to delivery via blood vessels that may not be sufficiently healthy or wide in size so as to allow delivery of both portions as a single unit.

One-Piece Valve Replacement Overview

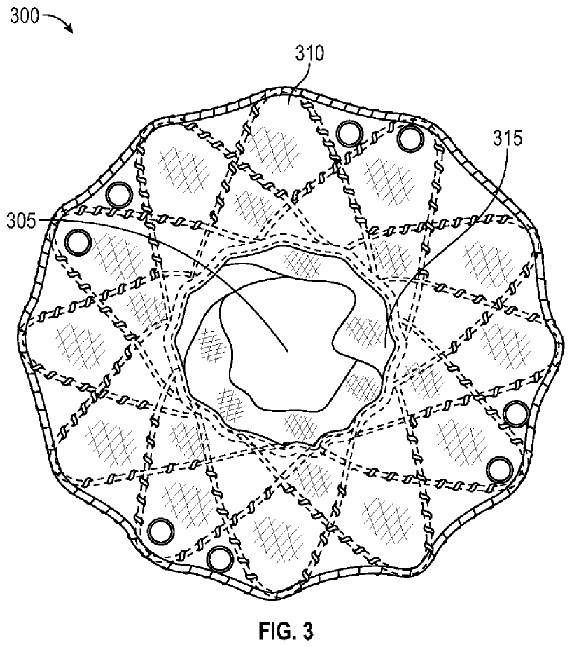

FIG. 3 generally illustrates an embodiment of a valve replacement as disclosed herein. FIG. 3 discloses an embodiment of a One-Piece System 300, comprising an opening 305 for blood flow, a sealing skirt 310, and a leaflet structure 315. Though not shown in FIG. 3, the One-Piece System 300 further comprises a body below the sealing skirt 310 that is similar to the body of the Adapter 200 in FIGS. 2A-2C and may further comprise anchors similar to anchors of the Adapter 200 in FIGS. 2A-2C. In one embodiment, the One-Piece System 300 may function as a permanent implant.

Whether as a One- or Two-Piece System, the Valve Replacement allows for valve-in-valve placement, wherein embodiments of the valve-in-valve placement comprise replacing existing leaflets and valve assemblies without a reduction in area (such as by placing new material over existing material), and without compromising the functionality of the implanted Valve Replacement.

Braided Structures

The braided structures disclosed herein are applicable to the One-Piece System and to the Adapter and the Valve Assembly of the Two-Piece System. Thus, though various embodiments of braided structures may be shown in relation to the Adapter and the Valve Assembly, it should be understood that such embodiments are also in relation to the One-Piece System.

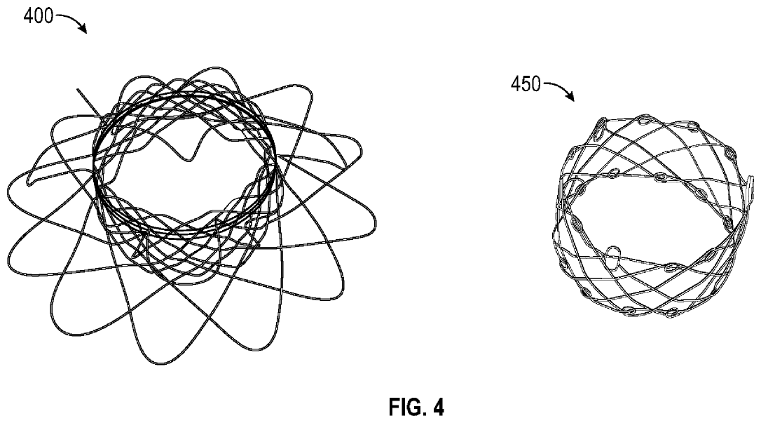

FIG. 4 generally illustrates an embodiment of a valve replacement as disclosed herein. FIG. 4 discloses the braided wire frame of an Adapter 400 and the braided wire frame of a Valve Assembly 450. The braided wire frame allows the Adapter 400 and the Valve Assembly 450 to be compressed, which, when released may expand in size. Similarly, the One-Piece System may also be compressed and expanded. The braided wire frame design thus enables the Valve Replacement to be compressed to a small diameter—such as 4 mm to 6 mm—such that it may be delivered in a catheter. The braiding of the wire and overlapping with other wires also reduces or eliminates fracturing of the wire because of the decreased stress on the frame. The braiding also enables various-sized wires to be used.

The braided wire frame of the One-Piece System, the Adapter 400, and Valve Assembly 450 may comprise various wire embodiments, such as a single wire, two or more wires (for example, grafted or welded together), and a wire spliced of multiple wires. The wire(s) making up the One-Piece System and the Two-Piece System may be constructed of varying material, such as nitinol, which has shape-memory characteristics, and vary in dimensions, such as in diameter size.

By integrating diverse wire thicknesses and braiding designs, the Valve Replacement conforms with various densities and characteristics (i.e., radial force and expansion) of the heart's anatomy. In this, the braided frame enables the Valve Replacement to have a flexible and conformable performance, wherein the Valve Replacement self-adapts and moves with the heart while being forgiving to anatomical anomalies—similar to the heart's helical structure, as will be disclosed herein. The braided frame also facilitates placement of the Valve Replacement, maximizes its seal, and prevents migration with an integrated and optimized anchoring system. The braided frame geometry of the Valve Replacement allows for diverse application, such as being customizable to mitral and tricuspid anatomies; allows for fewer sizes to be needed to treat most disease states; promotes rapid prototyping; allows incorporation of various design features; promotes quicker design advancement with rapid evaluation and optimization of features; and is scalable using conventional processes. The braiding structure also allows for more degrees of freedom and opportunities for the wires to be in various positions.

An embodiment of fabricating the braided wire frame comprises oversizing the braided wire frame in relation to heart valve, which allows for more radial force for the same amount of material and geometry, thus allowing the frame to open up more fully and function better. Furthermore, it decreases the manufacturing tolerances involved in manufacturing the Valve Replacement. Oversizing the braided frame biases the wire frame structure so that there is less motion between the wires as they are predisposed with elastic strain energy to conform and adapt with greater radial force. As a result, the valves have higher degree of consistency and the manufacturing tolerances associated with attaching the leaflets, for example, is greatly improved.

In one embodiment, the braided frame is wrapped and shape set such that it has enough radial force to self-expand and be opened up to desired radial capacity while still being configured to fit within a catheter.

Embodiments of the Valve Replacement may range in diameter from 25 mm to more than 55 pm. In one embodiment, the wire frame is oversized, which comprises braiding the wire frame on a mandrel that is 25.4 mm in diameter (or 28.0 mm or 32.0 mm, depending on the desired valve size) and shape-setting it by treating it in 505 degree C. salt/sand bath. The frame is then removed from the initial mandrel and stretched over a 29.0 mm mandrel (or 31.0 mm or 33.0 mm, e.g., for larger valves) and shape-set again. Temporary strings (or other similar methods known to one skilled in the art) are then run through the loops and tied using a 25.4 mm mandrel as a reference diameter for the valve frame. This compresses the frame by spring loading the loops (though other embodiments may comprise other structures beyond loops, such as simple apices). The braided Valve Replacement may thus be shape set at a larger diameter and then constrained to a smaller diameter and held with string until fabric is sewn onto the frame. In another manufacturing embodiment, the wire frame repeats a braid pattern over its length three times while wrapping five times around a circle.

Embodiments of the Valve Replacement may comprise compatibility with various-size catheters, such as 26F, 28F, 30F, 32F, and 34F.

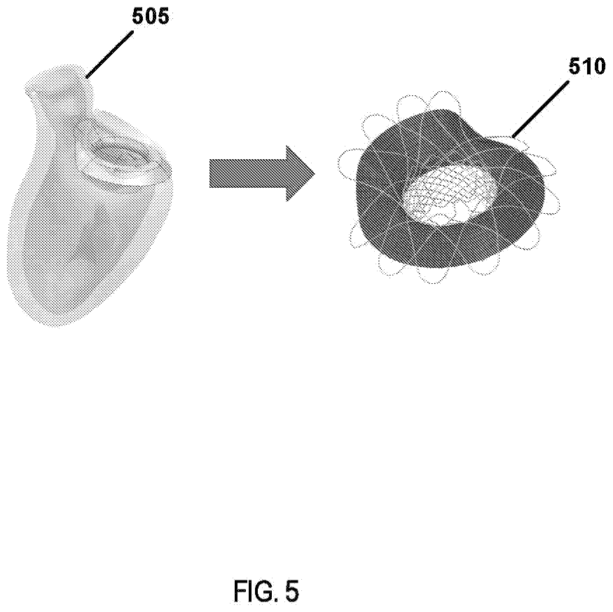

FIG. 5 generally illustrates an embodiment of a valve replacement as disclosed herein. As shown in FIG. 5, an Adapter 510 may be compatible with a human heart 505, wherein embodiments of achieving coaptation comprise sealing and anchoring by adjustment of the over-and-under pattern of the braid to realize separable sections of the braid that can behave independently. The Adapter 510 may be constructed of varying material and vary in dimensions. In one embodiment, the Adapter 510 may be made up of a nitinol wire braid of one or more wires with different diameters. When released the Adapter may expand in size (e.g., the body expanding to 25 mm or greater in diameter and the sealing skirt expanding anywhere from 40 mm to 70 mm in diameter).

The Valve Replacement may comprise other types of wire, such as stainless steel, cobalt chrome, and other types of implant metals. In other embodiments, the Valve Replacement may comprise polymer materials, such as biocompatible plastics and fiber-reinforced polymer. Some embodiments may comprise drawn-filled tubing (outside material NiTi and inside material some higher radiopaque material) for the Valve Replacement or portions of the Valve Replacement (e.g., anchors, or features desired to be seen under fluoroscopy). The Valve Replacement or portions of it may be made of hollow tubing. Additionally, flat wire or other cross sections of wire may be chosen for portions of the Valve Replacement, such as to provide tailored/increased stiffness for anchors.

Flanges and Anchors of the Braided Wire Frame

The Adapter is designed to preserve native ventricular filling by orienting flow into the ventricle in such a way as to limit turbulence and maximize efficient flow, such as towards the ventricle wall, between the papillary muscles, or otherwise oriented towards the apex of the ventricle (“virtual apex”).

The Adapter is also designed to be anatomically customized with patient and disease state-specific sizing. Sizing may be based on anatomical data, for example: Using a sizing tool to determine Adapter diameter and flange length, while also optimizing valve orientation for both ventricle outflow consideration and ventricular efficiency. In the example, parameters of the sizing tool are fed to the parametric device model, which automatically creates the pattern for the shape-set tooling.

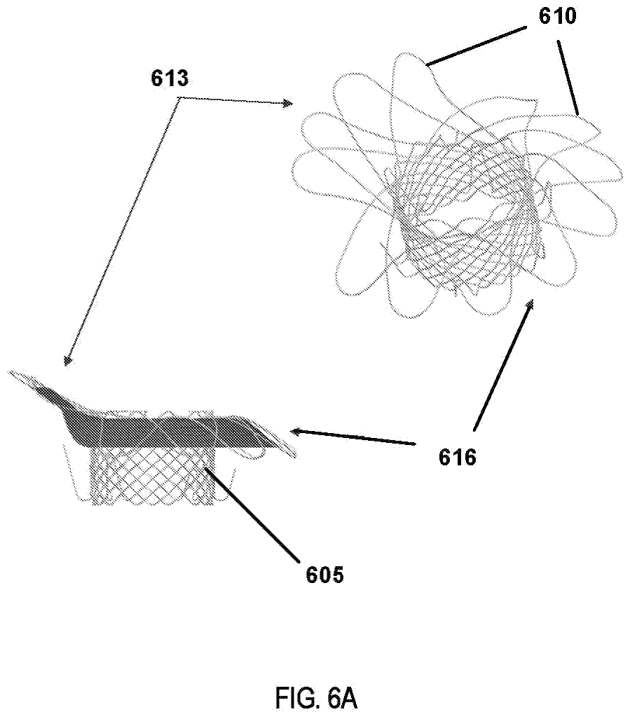

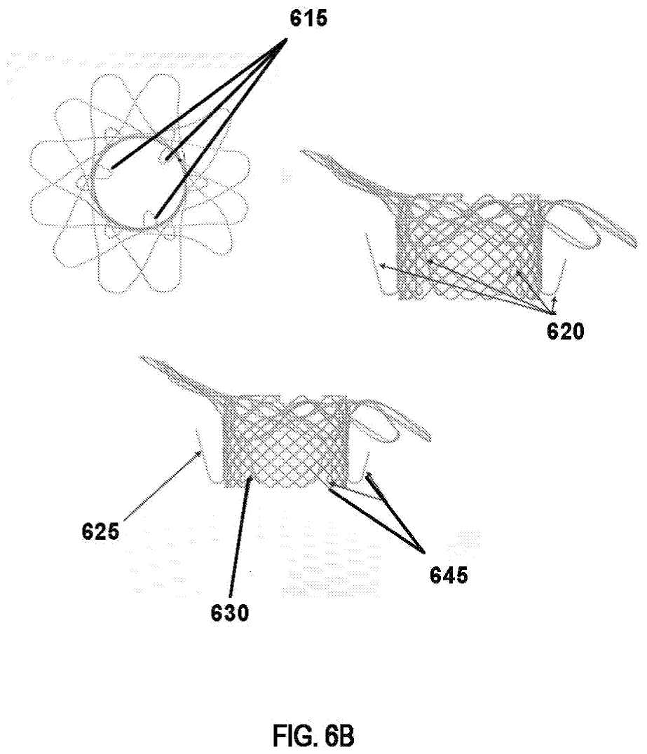

FIG. 6A generally illustrates an embodiment of a valve replacement as disclosed herein. As shown in FIG. 6A, the Adapter may comprise an Adapter body 605 and one or more atrial flanges 610. In one embodiment wherein the Adapter is applied to a valve, such as a mitral valve, the circumference of the atrial flange 610 is separated into one-third 613 and two-thirds 616. The one-third portion 613 of the atrial flange 620 engages with the fibrous aorta-mitral curtain and is formed at an angle that prevents the valve being pulled into the LVOT. This feature also maximizes sealing during systole. The two-thirds portion 616 of the atrial flange 620 engages the muscular wall and is formed at an angle that pulls the valve away from the LVOT and directs flow towards the apex of the ventricle, between the papillary muscles, or towards the ventricle wall. In other embodiments, the Adapter body and atrial flange function similarly or identically when applied to the tricuspid valve.

FIG. 6B generally illustrates an embodiment of a valve replacement as disclosed herein. As shown in FIG. 6B, the Adapter may comprise valve and retainers 615 within the inner frame of the Adapter body. The Adapter may also comprise sub-valvular anchors 620 for leaflet management. In one embodiment, the sub-valvular anchors 620 are made up of one or more of the following: anterior leaflet anchor 625, trigone anchor 630, and posterior leaflet anchors 645. For example, the Adapter may comprise a single anterior leaflet anchor 625, two trigone anchors 630, and three posterior leaflet anchors 645. The anchors may be configured to be biased in an upward direction so as to be radially overlapping in relation to the Adapter body.

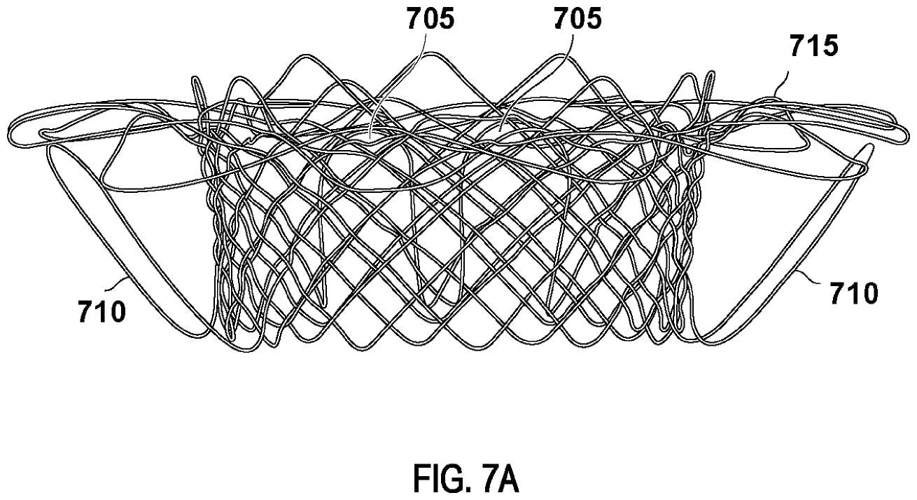

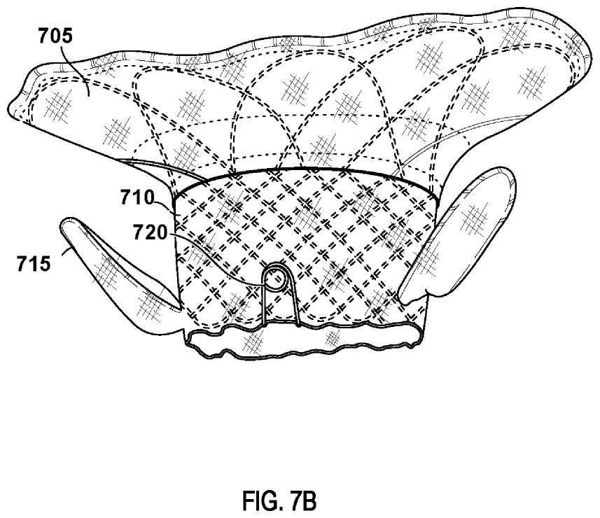

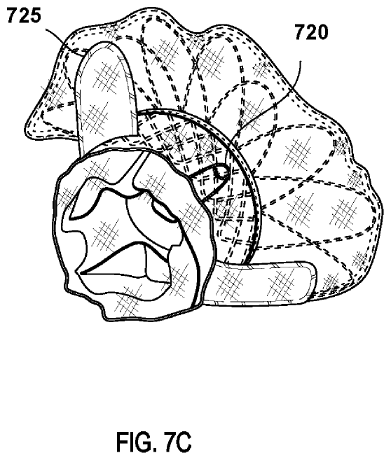

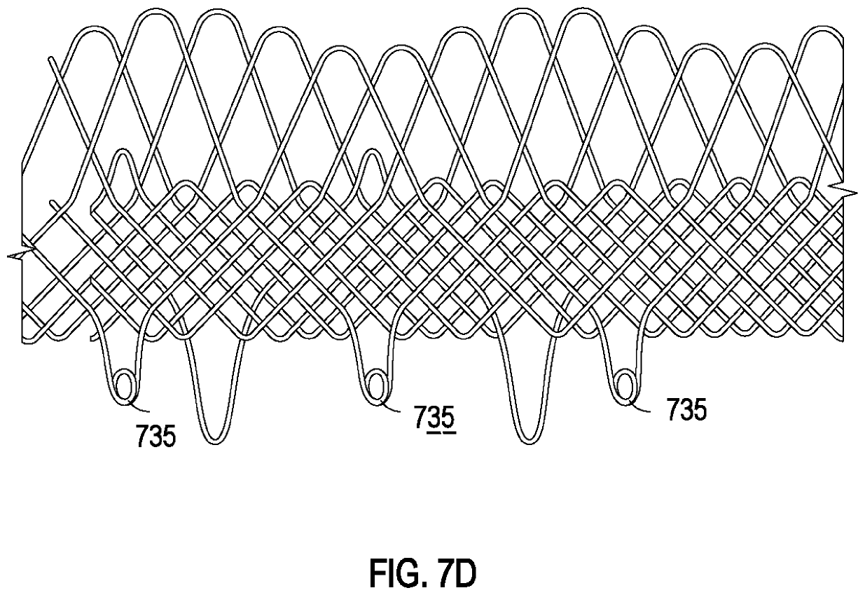

FIG. 7A generally illustrates an embodiment of a valve replacement as disclosed herein. FIG. 7A shows an embodiment of a wire braid frame that the Adapter is comprised of. The wire braid frame may comprise a 24-point braid pattern, with double posterior leaflet anchors 705, wherein the double posterior leaflet anchors 705 are used to maintain symmetry and additionally provide twice the structural anchoring. The wire braid frame may also comprise dual stabilization anchors 710. Also shown is that the wire braid frame may have the anchor locations available in 15-degree increments.

The anchors may be, in some embodiments, an extension of the tubular braided frame and extend out from the outflow end to function as an engagement attachment. In other embodiments, the wire braid frame of an Adapter may have anchors that are grafted, welded, or fused on. For example, FIG. 7A shows the combination of a larger gage wire (0.0175″-0.02″) (represented by the stabilization anchors 710) and smaller gage wire (0.012-0.0175″) (represented by the posterior leaflet anchors 705 and further represented by additional wires 715) by means of a joining operation at the interface between the varying-size wires. The connection interface may be a weld or a weld with a support tube.

Embodiments of welding used may be in relation to the material that the Valve Replacement is comprised of. In an embodiment of the anchors comprising a hollow tubing (hypotube) material, the inside diameter of the hypotube mates perfectly with the diameter of the wire so that a helical weld pattern may be used to join the anchor to the frame. There, the ends of the hypotube may be chamfered so as to present a smooth transition with the attached wire. Radiopaque wire may be inserted inside the hypotube and positioned to be at the peaks of the anchors (such embodiment provides optimal fluoroscopic visualization).

FIGS. 7B and 7C generally illustrate embodiments of a valve replacement as disclosed herein. In one embodiment, as shown in FIG. 7B, a Valve Replacement may comprise an atrial sealing skirt 705, a frame body 710, and a stabilization anchor 715 that are covered in a fabric for the purpose of flow sealing and/or encouraging (e.g., influencing either promoting or inhibiting) tissue growth after implantation. The embodiment may further comprise a clip 720 that is not covered in a fabric. FIG. 7C shows a Valve Replacement comprising posterior leaflet anchors 725 and clips 720.

FIG. 7D generally illustrates an embodiment of a valve replacement as disclosed herein. FIG. 7D shows an embodiment of the Valve Replacement comprising a clip component for the purpose of improving delivery control, via secure attachment of the Valve Replacement to a delivery catheter, and for the purpose of improving the efficiency and efficacy of leaflet attachment. FIG. 7D shows a flat-pattern schematic of a wire frame with a clip 735, wherein the clip 735 may be a looped portion of the wire frame extending out from the main body of the wire frame. In some embodiments, a clip 735 may be positioned at two or more separate locations around the circumference of the Valve Replacement. In other embodiments, clips 735 be shape set 180 degrees such that they can provide for a hook shape to clip onto the native valve leaflets. For example, once the Valve Replacement is released from a delivery system, the clips 735 may attach onto the native valve leaflets, providing securement of the Valve Replacement.

Various embodiments of the Valve Replacement may comprise various quantities of anchors. For example, one embodiment may comprise six anchors whereas another may comprise three anchors. In a preferred embodiment comprising three anchors, applicable to the mitral valve, the Valve Replacement comprises a 150° angle between the P1 and P3 anchors with the P2 anchor being symmetric between the P1/P3 anchors. In a preferred embodiment comprising three anchors, applicable to the tricuspid valve, the Valve Replacement comprises a uniform 120°/120°/120° spacing of the anchors.

The anchors may be made of the same wire as the braided frame or different wire—whether it be different in material and size. This provides a novel aspect: The ability to have thicker and/or more durable wire for the anchors allows for the anchors—which are required to attach to the valve tissue and maintain the Valve Replacement in place—to be stronger and/or firm, without comprising the flexibility of the body frame. This enables the Valve Replacement to remain firmly and securely positioned within the heart valve while still allowing the Valve Replacement to move and function in accordance with the heart's natural movements.

Another novel aspect is the synchronization between the flanges and the anchors. Once implanted, the flanges provide a downward force on the heart tissue as the anchors provide an upward force. These two forces exerted by the Valve Replacement further secure it in place without comprising the fluidity of the braided body frame or the functionality of the leaflets.

Helical Braided Design

The novel helical-braided designs of embodiments of the Valve Replacement purposefully leverage the natural helical movements of a beating human heart so as to balance both flexibility and strength. Studies of the human heart reveal that the mechanisms of ejection and suction are from a helical design of muscles in a “coil within a coil” formation, which are responsible for clockwise and counterclockwise rotation and functional activity. More specifically, the underlying anatomy of the human heart comprises a helical braid having a transverse basal loop of muscle for contraction that overlies an oblique helix that is responsible for ejection and suction within the heart.

The disclosed braided helical design is configured to put less stress on the individual components of the Valve Replacement because the Valve Replacement moves with the heart, i.e., the leaflets and anchors and other components have less stress and the Valve Replacement migrates less because its natural helical movement with the heart keeps it in in place.

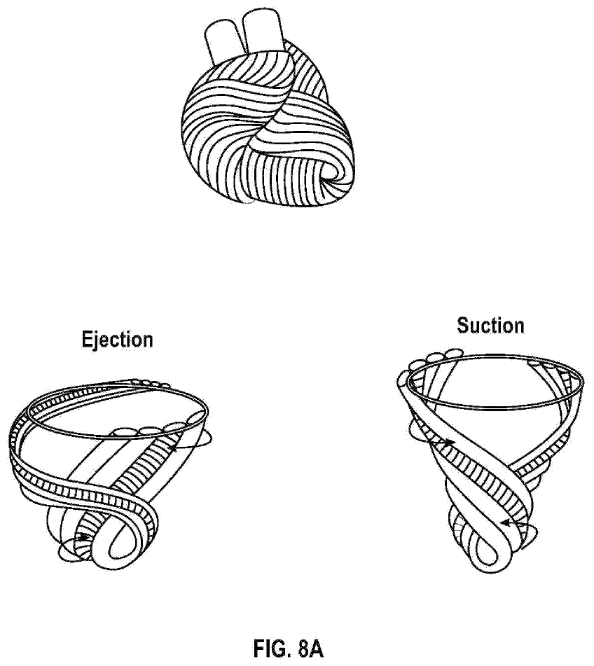

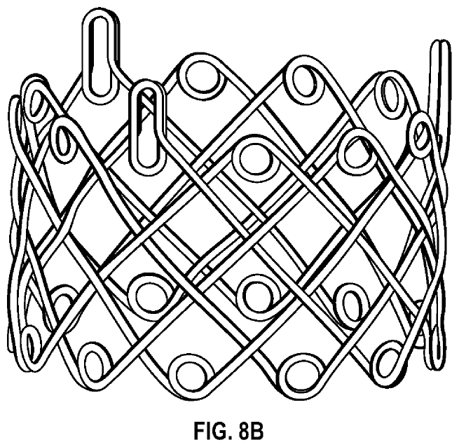

FIG. 8A generally illustrates the helical functionality of the human heart. As shown in FIG. 8A, the twisting and untwisting motions within the heart are created by inner helical spirals within the descending and ascending apical loop muscle segments, with the heart having a natural clockwise torsion/contraction for ejection and a natural counterclockwise loosening/lengthening for suction. In heart disease, the natural helix of the heart becomes architecturally altered in shape.

FIG. 8B generally illustrates an embodiment of a valve replacement as disclosed herein. As shown in FIG. 8B, an embodiment of the Valve Replacement comprises a helical braided design that mimics and reinforces the normal helical and elliptical formation of the heart and its twisting/turning motions. In one embodiment, the helical braided design comprises a design wherein the braided wires resemble a frame that is constantly turning in one direction as it is compressed and/or elongated around an open center. Both the One-Piece System and the Adapter and Valve Assembly of the Two-Piece System may comprise the helical braided design. A normal heart develops ejection and suction as a functional consequence of the contraction integrity of the apical ellipse. The braided helical design of the Valve Replacement maximizes shortening and lengthening of the heart muscles, thereby reinforcing the desired apical ellipse of a healthy heart movement.

For example, as the human heart muscles compress and descend, the braided helical wires of the Valve Replacement—rather than be stiff—also compress and descend with the heart muscles, thereby reinforcing a natural spiral compression and descension of the heart muscle surrounding the braided wires. With the braided helical design, the Valve Replacement conforms to and reinforces the natural movement of the heart. The braided helical design of the Valve Replacement produces a twisting spiral coil that develops torsion in a clockwise direction. And as the human-heart muscles lengthen and fill, the braided helical design reinforces a natural spiral lengthening and filling of the braided wires with the surrounding heart muscle, resulting in an untwisting spiral coil within the adapter or valve that develops an ejection force.

The novel braided helical design is significant for treating heart valves. By comprising a braided helical design, embodiments of the Valve Replacement reinforce the natural helical movement of the heart and more naturally adapt and sits within the desired valve area. For example, embodiments of the Valve Replacement will tend to remain in the desired mitral or tricuspid valve area because the braided helical design will move (contract, twist and shorten and untwist and lengthen) with the natural movements of the heart. This allows for the Valve Replacement to self-correct and seat within the valve area in a natural state, thus conforming to the heart's natural movements and encouraging central vortex flow.

The novel braided helical design thus facilitates a natural heart movement. In one embodiment, the Valve Replacement is held in place by the combined efforts of the flange and anchors, with the helical braided portion being in between the flange and anchors. The helical braided portion twists back and forth with the heart's natural movement, enabling a pumping-and-squeezing motion. The twisting motion, when the heart pumps, encourages flow of liquid through the Valve Replacement, thus allowing for better flow dynamics.

Material Covering

In some embodiments, different materials are prepared prior to assembling into a continuous covering. In other embodiments, material may be added and receive a modification treatment post-assembly that is applied to only specific locations on the Valve Replacement.

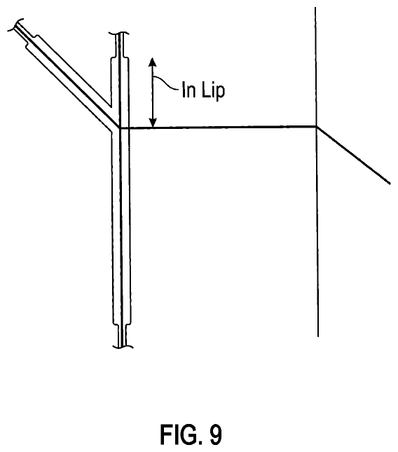

FIG. 9 generally illustrates an embodiment of a valve replacement as disclosed herein. As shown in FIG. 9, tissue attachment and ingrowth may be promoted in an area that is desired to become anchored to the tissue, while cellular interaction can be limited to simple endothelialization or no response at all, to allow disturbance of part of the device at a later date without risk of tissue or thrombus embolization. Put simply, the varying material used may be either conducive or non-conducive to chemical bonding. For example, in a preferred embodiment, the materials in contact between the inner portion of the Adapter and the outer portion of the Valve Assembly do not bond, so as to allow for movement of both portions; whereas the material on the outside of the Adapter bonds with human tissue. Thus, depending on the location, materials may be used such that cellular growth is inhibited or promoted.

FIG. 9 further illustrates how the Valve Replacement may be encased, either completely or partially, in a continuous material covering to elicit the type of physiological response that is desired as well as the mechanical behavior. Though the covering is continuous—as in there are no material gaps at the transitions of physical features—the materials may be modified locally in areas of the device to behave differently. For example, the material covering one side of the flange may deliberately be nonporous to facilitate sealing while the material on the other side of the flange may be a knit that facilitates tissue ingrowth for anchoring. Alternatively, the flange could be alternating rings of nonporous and ingrowth material on both sides of the flange. These techniques can be applied to any surface of the device.

Material differences range from being entirely different materials—natural tissue or synthetic fabric—to physical and chemical surface modification, to obtain the desired mechanical and biocompatible properties. These modifications can include but are not limited to coating, etching, mechanically biasing, ion infusion, various deposition techniques, and oxidizing/nitriding/carbiding. Modifications may be used in any combination to achieve the desired result.

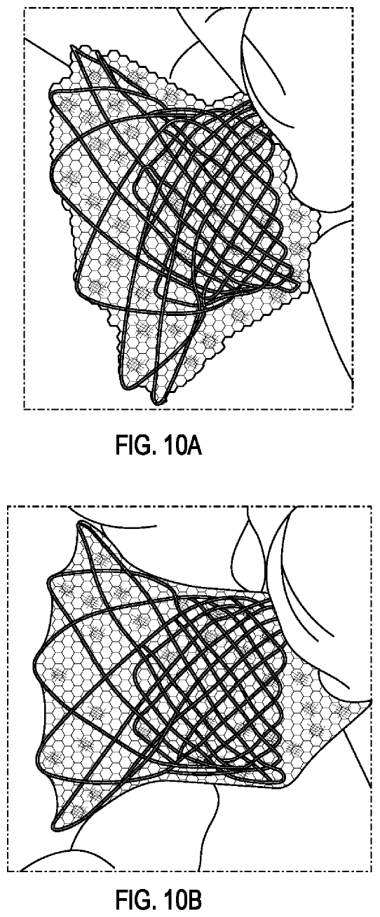

FIGS. 10A and 10B generally illustrate embodiments of a valve replacement as disclosed herein. As shown in FIGS. 10A and 10B, an embodiment of the Valve Replacement may comprise a continuous piece of material around the outside of the frame. A continuous seal may be configured from the material (such as fabric) extending from an inflow edge of the Valve Replacement to the extrados of the body of the Valve Replacement. A strip of ingrowth fabric may be sewn around the inflow edge of the Valve Replacement, with a non-porous coating forming a continuous seal extending into the ventricle.

The continuous surface of the fabric may be locally influenced and characterized for modulating or even contradicting properties, such as coating with medical polymer in locations where no tissue attachment is desired, hydrogels where space-filling or latent actions are desired, or a hydrophilic tissue adhesive. The continuous material structure of the fabric may be voluminous in nature, filling space and adapting the round heart valve to the asymmetrical shape of the valve annulus. Combined with other attachment methods, an embodiment of the mitral-valve adapter fabricated with this method aids in engagement and attachment of the leaflet tissue and other sub-valvular structures. The partially porous fabric provides an improved seal for a replacement valve, enabling accommodation to irregular shaped anatomy through the compliance of the fabric.

In other embodiments, the Valve Replacement may be fabricated using a constraint to hold the Valve Replacement at a specific dimension while attaching material to influence device performance. A fabrication technique is disclosed, which acts to influence the disposition of a braided wire frame—removing the inherent freedom of movement and unpredictability that is present between relative members of the frame structure when in a load-free state. This technique involves restraining the radial expansion of the frame with a constraint, such as feeding some number of sutures through or around the structure to hold it at a specific dimension other than its unrestrained, “free” dimension. In subsequent fabrication steps, the structure is incorporated into an assembly that adopts this new configuration and considers this to be the final dimension. When the constraints are removed from the braided frame, this braided frame tries to recover to its original “free” dimension—applying additional radial force to the surrounding structure while being constrained to the desired dimension.

The degree of radial force transmitted to the fabric material from the frame can be adjusted as required to achieve the optimal combination or performance properties. In particular, the strain energy density of the structure can be more uniform. A greater stiffness is achieved (resulting in a better seal) with less material, resulting in a more low-profile structure. The suture finally provides a biasing of the structure toward a desirable diameter and height for the valve structure.

To expand the concept further, structures that possess features described herein may be co-deployed singularly or with a connected design, so as to engage both the mitral and the aortic valve apparatus and/or annulus. The intent is to influence the leaflets of both valves, as well as the angulation of the valves relative to one another, to ensure the most effective management of flow through the ventricle and maximizing the efficiency of the outflow tract.

In some embodiments, the Valve Replacement is covered in a material that wraps around the frame in a continuous manner. Embodiments of the material are fabric and animal tissue. By using materials that can be locally modified to change characteristics such as porosity and surface roughness, a certain level of control over cellular interaction on the various parts of the device can be achieved. In other embodiments, the Adapter body and atrial flange may be covered in fabric for the purpose of flow sealing and/or influencing (e.g., either promoting or inhibiting) tissue growth after implantation.

The material used further assists with the loading and deployment of the Valve Replacement. For example, the material may promote the Valve Replacement to function as a re-valve system, wherein a tubular braided fabric tube (coated with a polymer to decrease porosity to blood) surrounds the frame and constrains the diameter. This tube is sewn onto the frame, sometimes in conjunction with a leaflet panel, so that the strings can be removed and what remains is a pretensioned frame constrained by the fabric.

Sewing Methods and Belt Loops

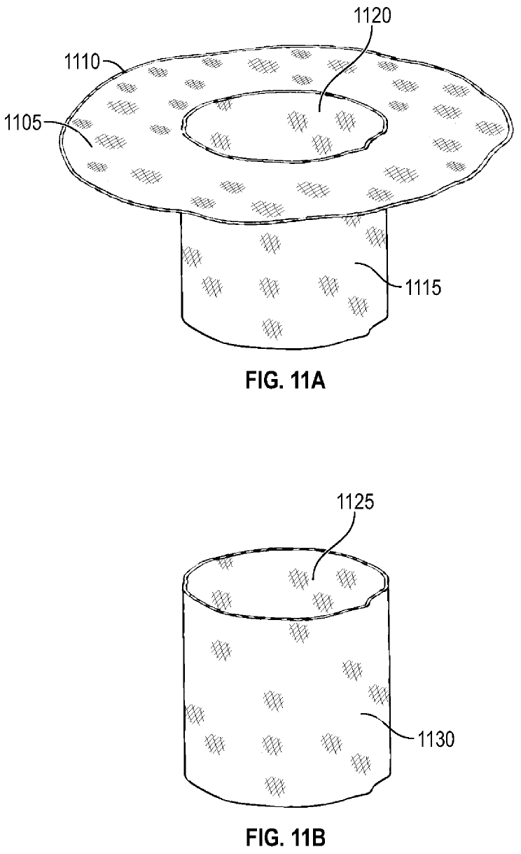

FIGS. 11A and 11B generally illustrate embodiments of a valve replacement as disclosed herein. More specifically, FIGS. 11A and 11B show various embodiments of the fabrication of material for the Valve Replacement, focused on the Two-Piece System.

FIG. 11A shows an aran flange 1105, a secant flange 1110, a secant outer cuff 1115, and an aran inner cuff 1120 of the Valve Replacement. The aran flange 1105 comprises a circular top piece, wherein it may further comprise a coating to reduce fluid permeability. The secant flange 1110 comprises a circular bottom piece that stretches to allow expansion of the Valve Replacement when deployed. The secant outer cuff 1115 comprises a cross-stich that attaches the ends to create a tube-like shape. The aran inner cuff 1120 comprises a running or cross-stitch that attaches the ends to create a tube shape, wherein the fabric of the aran inner cuff 1120 has limited elasticity and either stitch maintains integrity of the aran inner cuff 1120.

In one embodiment, the secant flange 1110 and the secant outer cuff 1115 may be combined, either by cross-stitching or other method know to one skilled in the art, to form a secant bottom piece. The flange may be stitched furthest away from any anchor slots. The formed secant bottom piece may be positioned onto the bottom of the Adapter or the One-Piece System, wherein the shape-set thread around the body of the frame is cut and wrap-stitch is used to close or secure the anchor slots.

In another embodiment, the aran inner cuff 1120 may be attached to the inner opening of the aran flange 1105 to form an aran top piece. The aran top piece may be slide over the top of the Adapter or the One-Piece System after which the fabric is wrap-stitched closest to the frame.

In another embodiment, the secant bottom piece and the aran top piece may be connected, such as by wrap stitching at the bottom of the frame to connect the aran top piece to the secant bottom piece. For this, the fabric of the aran flange 1105 and the secant flange 1110 may be smoothed and held in place (such as with sewing clips) and connected around the wire flange (such as with a running stitch), wherein the border of the flange may be circular and not rigid. The excess fabric may be trimmed, and additional stitch may be added around the flange and each wire flange tip. Additional stitching may be performed along the wires to secure the fabrics together and keep flush against the wire flange. The stitching may be done to the second crossing of wires, followed to the next wire, and then up towards the tip of the wire flange. This process may be continued around the flange and repeated on the next set of wires.

In one embodiment of the secant outer cuff 1115, the secant outer cuff 1115 may be folded in half and secured together (such as with a sewing clip), after which a blanket stitch may be performed around the edges. The blanket stitch allows the secant outer cuff 1115 to retain its shape without sinching the fabric. The secant outer cuff 1115 may be turned inside out and placed over the anchor wire, after which the secant outer cuff 1115 is secured to the front and back of the anchor wire (such as with a running stitch). In this, the seam of the stitch (used to close the anchor slots) may be caught between the front and back fabric of the secant outer cuff 1115 to secure it to the Adapter or One-Piece System. The running stitch may encompass the front of the secant outer cuff 1115, the seam, and the back of the secant outer cuff 1115 along the base of the anchor wire. Once the secant outer cuff 1115 is secured at the base, a stitch may be continued along the anchor wire to keep the secant outer cuff 1115 from slipping or sliding on the anchor. Fabric may be slightly caught, wherein it is not loose enough to leave excess fabric but not tight enough to affect the shape of the wire.

FIG. 11B shows an aran inner valve cuff 1125 and an aran outer valve cuff 1130. In one embodiment, leaflets may be connected to the aran inner valve cuff 1125, such as along a strip of fabric with a double running stitch along the belly of the leaflet, wherein the stitches are uniform across the leaflets to allow for proper valve opening and closing. The leaflet tabs may be exposed, such as by laser-cutting with slots at the top of the aran inner valve cuff 1125 (wherein a strip of the aran inner valve cuff 1125 may be folded in half, making sure that leaflets are aligned on top of each other; and wherein the ends of the aran inner valve cuff 1125 may be attached to the junction of the belly and tabs with a double running stitch). Following the exposure of the leaflet tabs, the aran inner valve cuff 1125 may be placed inside the Valve Assembly frame, the tabs may be pulled through commissure wires and laid flat between the leaflets and the aran inner valve cuff 1125.

In one embodiment, the ends of the aran outer valve cuff 1130 may be connected and the aran outer valve cuff 1130 slide over the outside of the Valve Assembly frame.

In another embodiment, the arran inner valve cuff 1125 and the aran outer valve cuff 1130 may be connected together. After placing the arran inner valve cuff 1125 on the inside of the Valve Assembly frame and the aran outer valve cuff 1130 on the outside of the Valve Assembly frame, the two parts may be connected to the bottom of the frame (such as by tacking down both parts with a square knot) directly below commissure wires and the parts may be stitched along the bottom of the frame. Following a commissure attachment, which is set forth in the following paragraph, both portions may be combined by sewing through the frame and the top of the Valve Assembly frame may be stitched. Additional steps may comprise, along the upper perimeter of the valve, stitching around the wires travelling from the commissures downwards and away from the peaks so as to create a z-shaped pattern. In this, the stitches may connect the inner fabric behind the leaflet and the aran outer valve cuff 1130.

In an embodiment of a commissure attachment, leaflet tabs are fed through the commissures, wherein each tab folds towards its own leaflet and is wrapped around the commissure wires. The ends of the tabs may be held together against the entry of the tabs and secured together, such as with running stitches vertically and on the inside of the Valve Assembly. Stitching may continue in front and around the commissure, such as for 3-4 times, and entering and existing at the location of the running stitch. Stitches may be perpendicular, comprising of embodiments such as a running stitch along the y-axis and a wrap stitch along the x-axis.

In other embodiments of the fabrication of material for the Valve Replacement wherein the focus is on the One-Piece System, a fabric for the secant portions comprises a stretchy and semi-transparent fabric; wherein the secant outer cuff 1115 and secant flange 1110 are sewn together to create the outer piece. Cross stitch may be used to connect the edges of the secant outer cuff 1115 and to connect the secant outer cuff 1115 to the secant flange 1110.

In a separate embodiment, a fabric for the aran portions comprises an inflexible and opaquer fabric where the coating is visible; wherein the aran inner cuff 1120 and aran flange 1105 are sewn together to create the inner piece. A running stitch may be used to connect the edges of the aran inner cuff 1120 and a cross stitch is used to connect the aran inner cuff 1120 to the aran flange 1105.

For these embodiments focused on the One-Piece System, an aran inner valve cuff may be created, wherein leaflets are attached to the aran inner cuff 1120 and wherein leaflet tabs are placed through slots of the aran inner cuff 1120 where the junction of the aran inner cuff 1120 and tabs meet, with the leaflets held in place, such as with sewing clips. Using a double running stitch, the belly's edge of each leaflet is sewed to the aran inner cuff 1120. The tabs and top edge of the leaflet(s) are flushed and level with one another and the running stitch on each belly of the leaflet(s) is level and uniform. (Inconsistent stitches can lead to a defective valve.) After the leaflets are attached to the aran inner cuff 1120, the aran inner cuff 1120 is folded in half, keeping leaflets level and held in place. A double running stitch may then be sewn directly down from the junction of the leaflet tabs, continuing away from the leaflets with a running stitch back up towards the junction of the leaflet tabs. (The running stitch should be away from the leaflet belly.) Following these steps, the aran inner valve cuff should create a tube.

An aran set may be created by connecting the aran inner valve cuff from above to the aran flange 1105, such as with a cross stitch, wherein leaflets are away from the seam.

Once the aran set is created, it may be connected to the secant set (the secant flange 1110 and secant outer cuff 1115 previously sewn together) by using a running stitch through the frame (between the double running stitch of the leaflet belly) and following the belly stitch of the leaflet(s) to secure both sets together. The tabs are then secured through the commissures and wrap stitch is used to connect the secant set to the aran set at the base of the frame. The deployment apertures are created, by cutting the fabric, before finishing the wrap stitch. Once connected, a beta stitch is incorporated on the flange and anchors cuff placements.

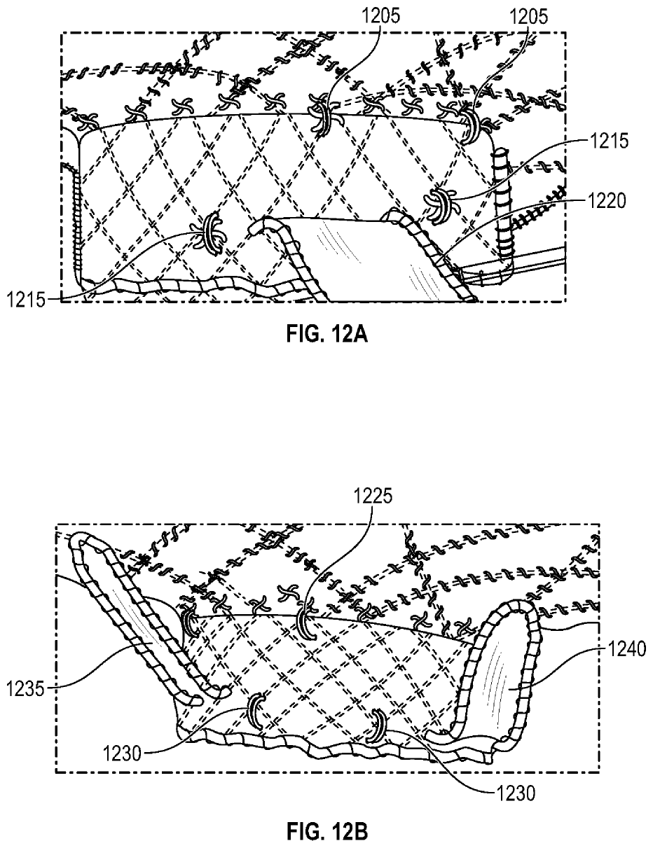

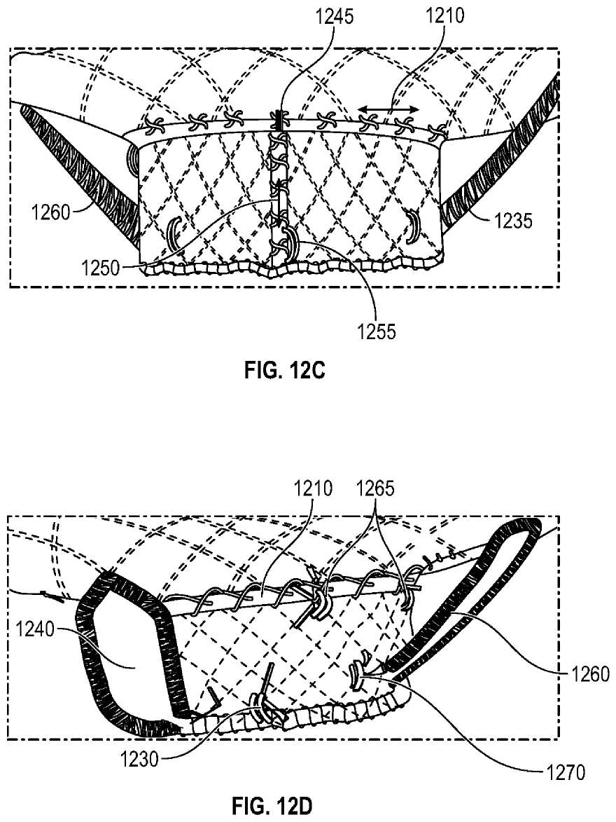

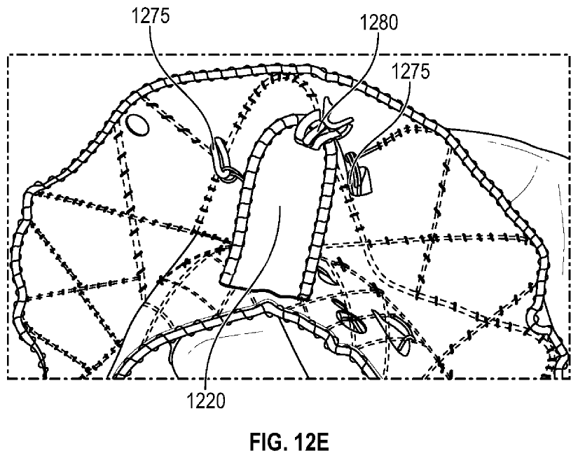

FIGS. 12A-12E generally illustrate embodiments of a valve replacement as disclosed herein. More specifically, FIGS. 12A-12E illustrate deployment belt loops commonly used for the Two-Piece System. The deployment belt loops may comprise one or more sets of loops.

In a preferred embodiment, the Adapter comprises seven belt loops at the base of the body of the Adapter, wherein there are belt loops on either side of three anchors and one belt loop at a vertical seam. The Adapter further comprises five belt loops at the horizontal seam between the body and the flange, wherein the five belt loops are located at cross wires on the same plane as one another. Hidden belt loops may be found behind the long anchors (P1/P3 anchors). The Adapter also comprises four belt loops along the flange, located at the crossed wires to the left and right of the long anchors. Additionally, the Adapter comprises two belt loops at the tips of the long anchors. Though in a preferred embodiment the anchor-tip belt loops comprise three loops and all other belt loops comprise two loops, it should be known that the belt loops are not limited to a specific number of loops.

FIG. 12A shows the belt loops in relation to the P1 and P3 anchors. The Adapter comprises one or more belt loops 1205 behind (i.e., on the body and hidden from view unless the anchor is lifted up) the P1 and/or P3 anchor 1220 and along the horizontal seam 1210. And the Adapter comprises and one or more belt loops 1215 to the left and right of the P1 and/or P3 anchor 1220.

FIG. 12B shows the belt loops in between the P1 1235 and P2 anchors 1240. Belt loops 1225 along the horizontal seam 1210 are at the same plane. The Adapter further comprises belt loops 1230 to the side of the P1 anchor 1235 and the P2 anchor 1240, wherein the belt loops 1230 are also approximately at the same plane.

FIG. 12C shows the belt loops in between the P1 1235 and P3 anchors 1260. One belt loop 1245 is at the junction of the horizontal 1210 and vertical seam 1250 and one belt loop 1255 is at the bottom of the vertical seam 1250.

FIG. 12D shows the belt loops in between the P2 1240 and P3 anchors 1260. The belt loops 1265 along the horizontal seam 1210 are at the same plane. Because the P3 anchor 1260 is slightly higher, the belt loop 1270 near the P3 anchor 1260 is adjusted to a height approximate to the P3 anchor 1260. The belt loop 1230 is in a position in relation to the location of the P2 anchor 1240. In a preferred embodiment, the anchors are biased towards the flange. In other embodiments, the anchors may be perpendicular to the body.

FIG. 12E shows anchor loops in relation to the P1 and/or P3 anchors. In one embodiment, the anchor loops comprise double loops 1275 positioned at the crossing wires on the flange to the left and right of the P1 and/or P3 anchor 1220, and the anchor loops further comprise a triple loop 1280 at the tip of the P1 and/or P3 anchor 1220.

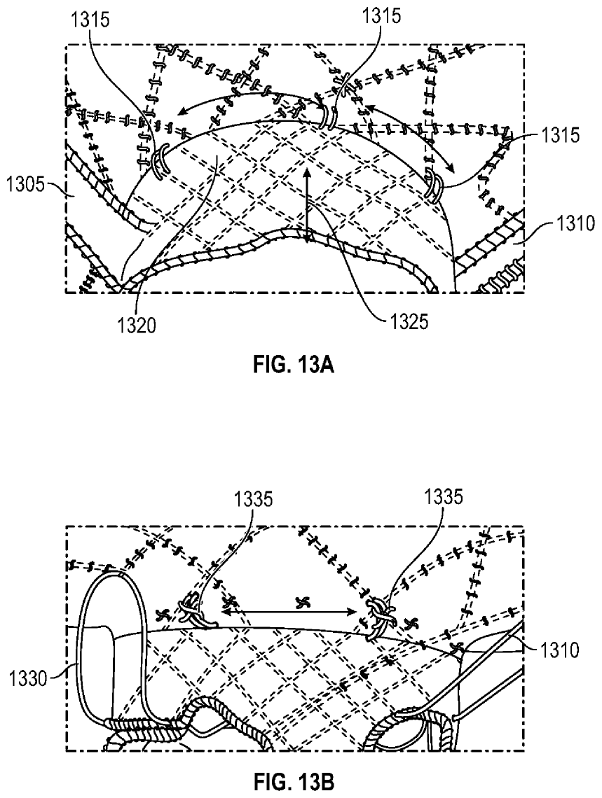

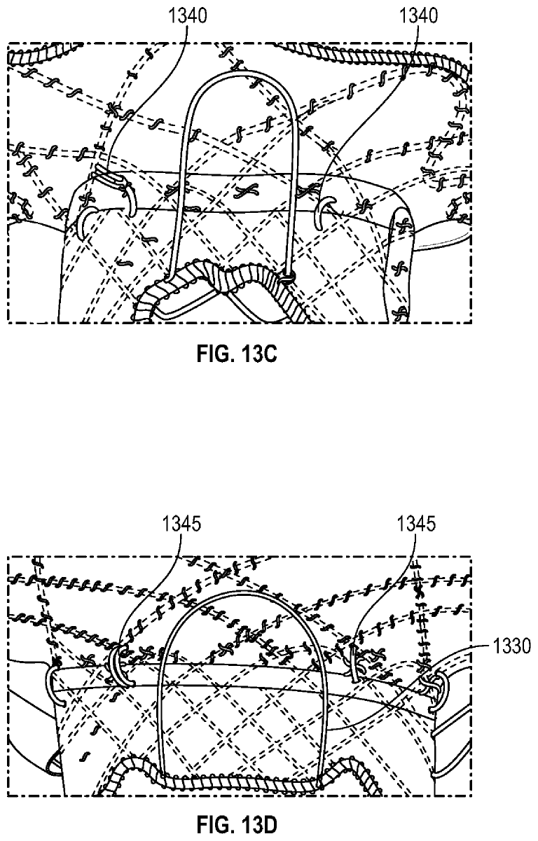

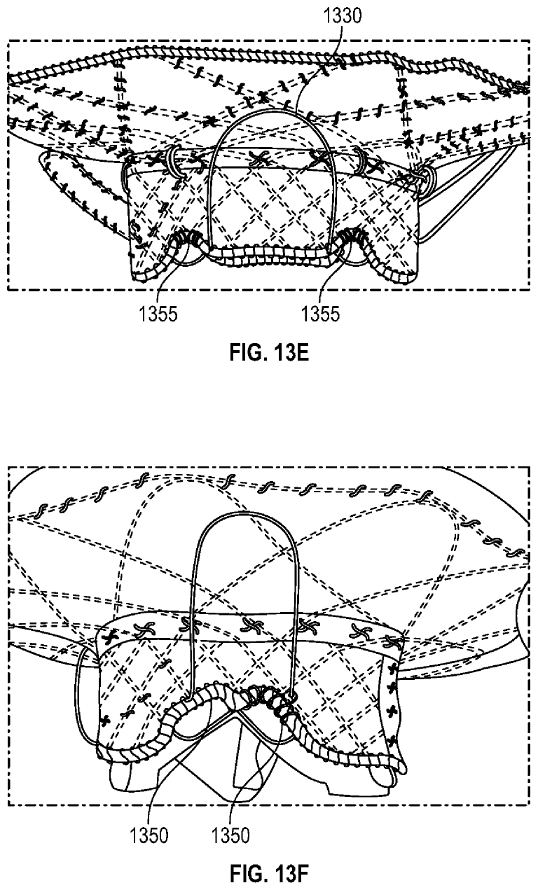

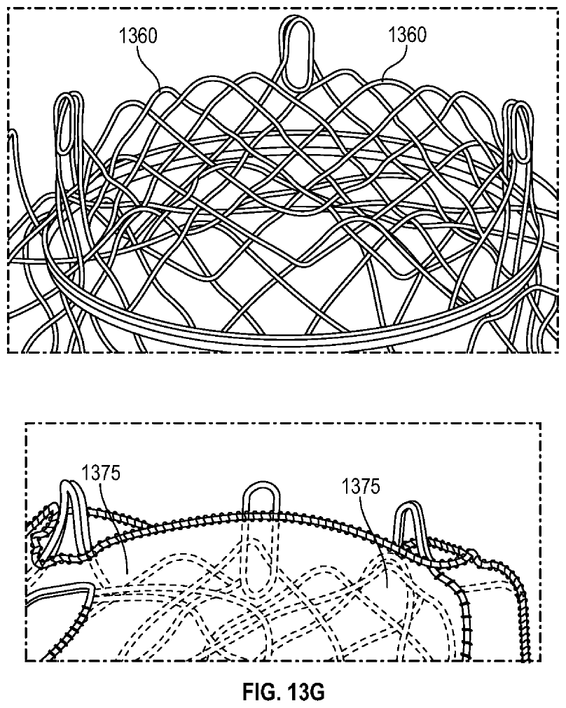

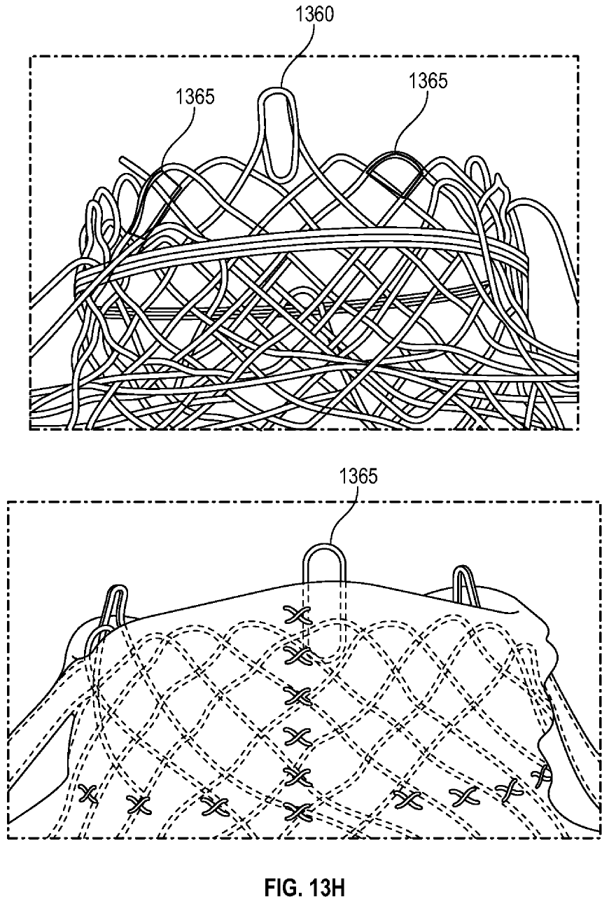

FIGS. 13A-13D generally illustrate embodiments of a valve replacement as disclosed herein. FIGS. 13A-13D illustrate deployment belt loops commonly used for the One-Piece System. The deployment belt loops may comprise one or more sets of loops.

In a preferred embodiment, the One-Piece System comprises seven belt loops, wherein the belt loops on the adapter body of the One-Piece System are double loops and the belt loops located on a horizontal seam are at the crossed wires along the same plane.

FIG. 13A shows belt loops in between the P1 1305 and P3 anchors 1310. Three belt loops 1315 are located along the horizontal seam 1320 with one belt loop located at the junction of where the horizontal 1320 and vertical seams 1325 meet and located in line with a commissure wire. The two other belt loops are located just outside of the P1 1305 and P3 anchors 1310.

FIG. 13B shows belt loops in between the P2 anchor 1330 and either the P1 1305 or P3 anchor 1310. Two loops 1335 are located along the horizontal seam and secured at the same plane of the crossed wires outside the P2 anchor 1330 and the long anchor (P1/P3 anchor).

FIG. 13C shows the belt loops 1340 of the long anchor, wherein the belt loops 1340 are located on either side of the long anchor. FIG. 13D shows the belt loops 1345 of the P2 anchor 1330, wherein the belt loops 1345 are located on either side of the P2 anchor 1330.

FIGS. 13E and 13F show an embodiment of the location of deployment apertures, wherein the deployment apertures 1350 are located directly below each long anchor and are located on either side 1355 of the P2 anchor 1330.

FIGS. 13G and 13H show another embodiment of the location of deployment apertures 1375. In this embodiment, fabric is removed (e.g., in a triangular shape) two peaks away 1360 from the commissures 1365 so as to expose the wire peak. A wrap stitch 1370 is placed around the perimeter of the frame so as to catch wires around the exposed peaks.

Engagement Structures

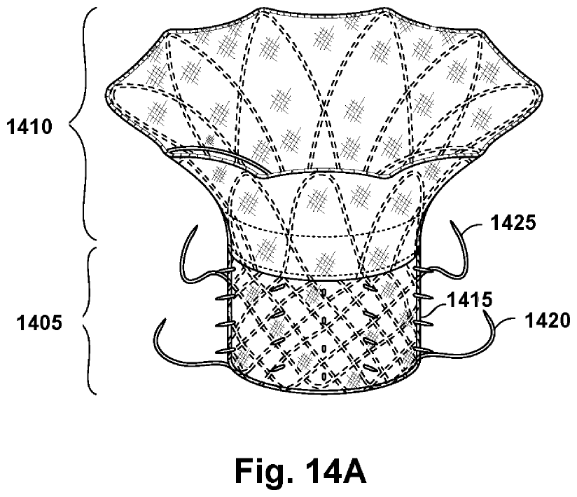

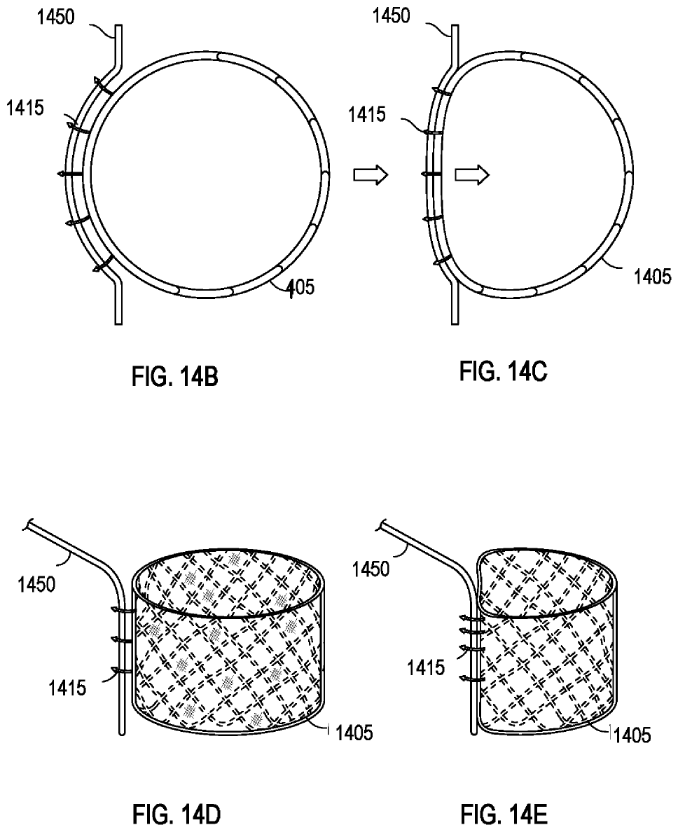

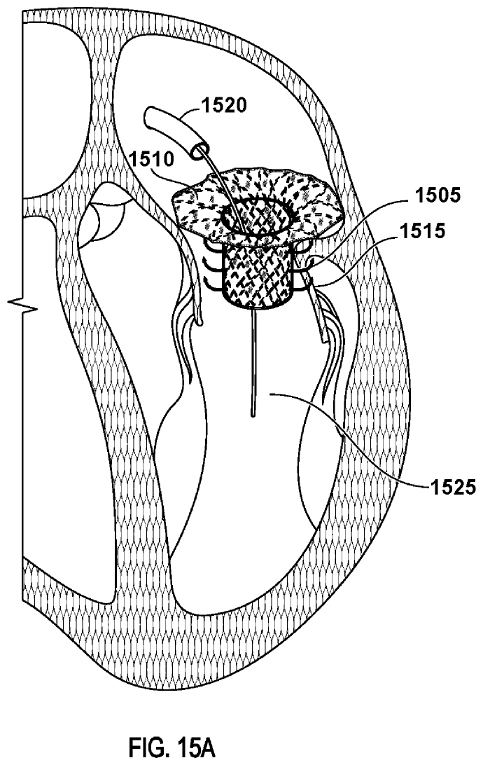

FIG. 14A generally illustrates an embodiment of a valve replacement as disclosed herein. In an embodiment, as shown in FIG. 14A, the Adapter or the One-Piece System comprises a body 1405 and an atrial sealing skirt 1410. The Adapter body 1405 and sealing skirt 1410 may be constructed of varying material and vary in dimensions. For example, the Adapter body 1405 and sealing skirt 1410 may be made up of a wire braid of one or more wires with different diameters. The wire may be made of material such as nitinol and designed to be compressed to a small diameter—such as 4 mm to 6 mm—to be delivered in a catheter. When released the Adapter body 1405 and sealing skirt 1410 may expand in size (i.e. the body expanding to 25 mm or greater in diameter and the sealing skirt expanding anywhere from 40 mm to 70 mm in diameter).

The exterior surface of the Adapter body 1405 may also be covered with a multitude of small, short barbs 1415. The barbs 1415 may be used to engage the leaflet or annulus of a malfunctioning cardiac valve, such as a mitral valve. The barbs 1415 may be made up of basic, short wires and/or may also have an extra barb-component, like a fishhook barb, to fixably retain the annular tissue.

The Adapter body 1405 may also have one or more hooks 1420 or 1425 (more or less in number than the barbs 1415) varying in size, that can hook under the native valve tissue. These larger hooks may or may not have fishhook barbs. The larger hooks may have a spring-like function that engage with the native valve tissue and prevent it from moving.

In a preferred embodiment, the sealing skirt 1410 may be connected to a catheter, wherein the Adapter Attachment is sequentially released from the catheter once the Adapter body 1405 is released and engaged with annual tissue. The sealing skirt 1410 may be designed to flex downward, toward, or even past the plane defining the joint between the Adapter body 1405 and the sealing skirt 1410—so as to be radially overlapping with the Adapter body 1405. The multitude of barbs 1415 on the Adapter body 1405 would work together to ensure the Adapter body 1405 is strongly engaged in the native annulus and resists the downward pressure of the sealing skirt 1410, such that the sealing skirt 1410 would create a strong seal against the atrial tissue surrounding the native valve annulus.

FIGS. 14B-14E generally illustrate embodiments of a valve replacement as disclosed herein. In these figures, the sealing skirt is not shown for ease of illustration. As shown in FIG. 14B, the Adapter body 1405 is designed with a braid of varying weave densities and/or wire diameters, and/or combined with releasable mechanisms such that the Adapter body initially has a round cross-section. The Adapter body 1405 has barbs 1415 designed to engage a native valve leaflet 1450. Once the barbs 1410 are engaged, the anchoring/attaching functionalities of the Adapter Attachment cause it to conform to a “D-shape” or other asymmetrical shape—keeping the Adapter body 1405 cylindrical or otherwise specifically shaped to receive the valve structure. This accommodation and conformity is achieved via the different weave, wire diameters, or mechanism enabling such. As shown in FIG. 14C, the change in shape creates a sharper curve radius to make the D-shape. The change from a circular cross-section to a D-shape cross-section may pull the leaflet, which can be useful, for example, in a mitral valve where an implant such as the Adapter body may cause outflow tract obstruction. FIGS. 14D and 14E disclose an oblique view of the structure and mechanism corresponding with FIGS. 14B and 14C. Embodiments disclosed in FIGS. 14B-14E may also comprise the sealing skirt and other features described in previous drawings.

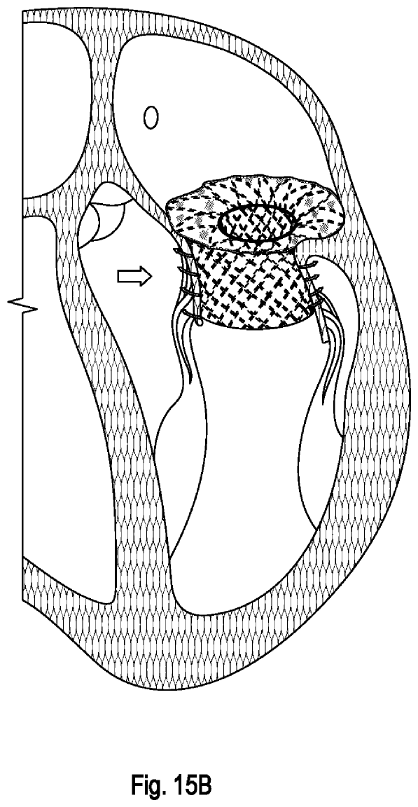

FIGS. 15A and 15B generally illustrate embodiments of a valve replacement as disclosed herein. FIG. 15A discloses an embodiment of the Valve Replacement implanted in a malfunctioning mitral valve, with the body 1505 deployed in the mitral valve and the sealing skirt 1510 deployed against the floor of the left atrium. In this embodiment, the Adapter body 1505 is oriented at a slight angle (i.e., from 10-30 degrees relative to the plane of the skirt), such that when deployed, the Adapter body 1505 is biased towards the posterior leaflet 1515.

Deployment as disclosed in FIG. 15A ensures good engagement of barbs into the posterior leaflet but not necessarily the anterior leaflet. The system may be designed to normally be in this geometric condition but be mechanically expandable by design so that it can expand to engage the anterior leaflet, then released back to the normal position after the barbs and/or hooks engage the anterior leaflet. This forces the anterior leaflet towards the posterior leaflet and away from the LVOT, ensuring it is not obstructed post procedure. Also shown are the delivery catheter 1520 and a guidewire 1525.

In another embodiment, the body of the Valve Replacement may be used to engage the leaflets with the barbs, wherein the body expands to a diameter larger than the diameter at deployment to ensure engagement with the leaflets. As the device is further deployed, the diameter of the engaged portion reduces to a final configuration—symmetrical or asymmetrical—thereby pulling the leaflets towards the device and away from the LVOT.

FIG. 15B generally illustrates an embodiment of an Adapter attachment as disclosed herein. FIG. 15B discloses a final configuration of an Adapter in its original position after release, wherein the anterior leaflet is drawn and held towards the posterior leaflet, ensuring no obstruction of the LVOT.

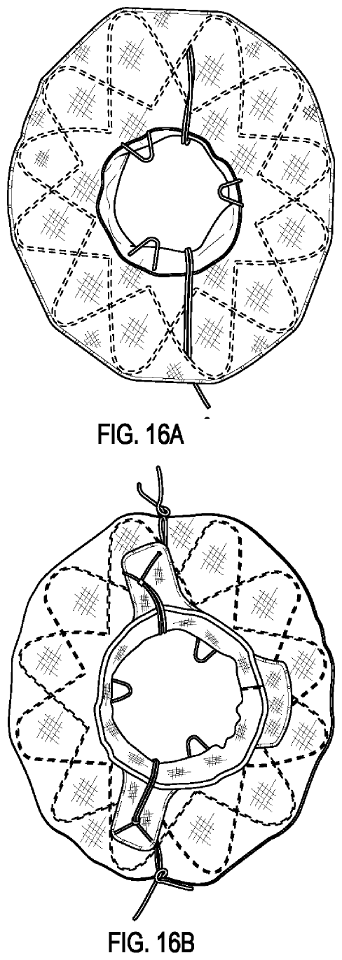

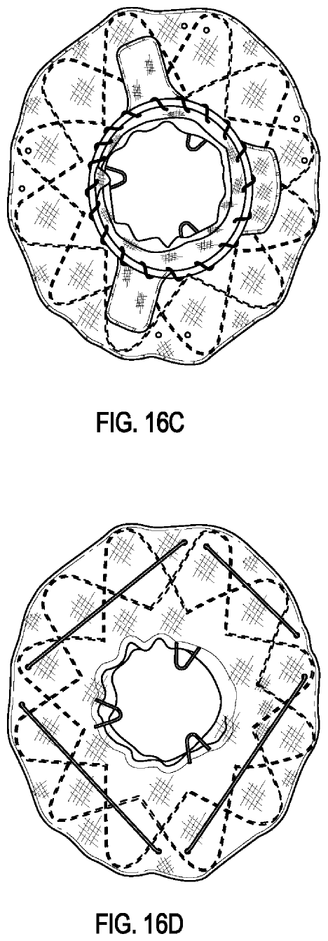

FIGS. 16A and 16D generally illustrate embodiments of a valve replacement as disclosed herein. FIG. 16A shows a top view of a Valve Replacement and FIG. 16B shows a bottom view of the Valve Replacement. As shown in FIGS. 16A and 16B, the inflow end of the Valve Replacement may comprise anchor retracting chords coming through the flow portion and anchor to the underside of a flange. These sutures permit control of the anchors by pulling and releasing the chords. Alternatively, the sutures may be releasably attached to a delivery system to provide similar manipulation of the anchors. FIG. 16B further discloses the chords attached to the anchors. FIGS. 16C-16D show attachment configurations for a collapsible flange.

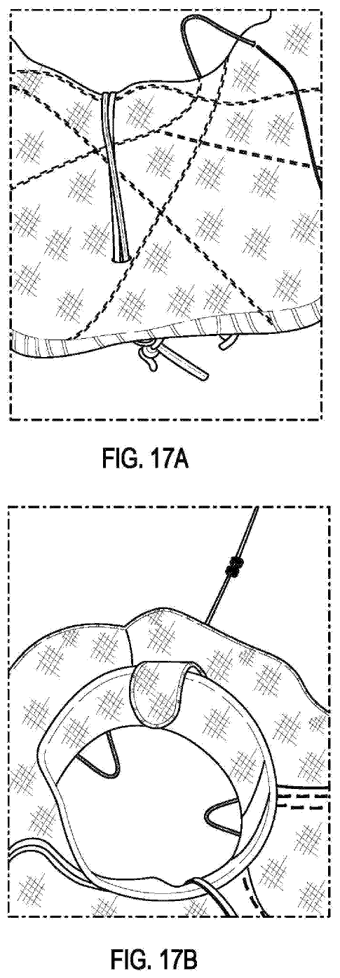

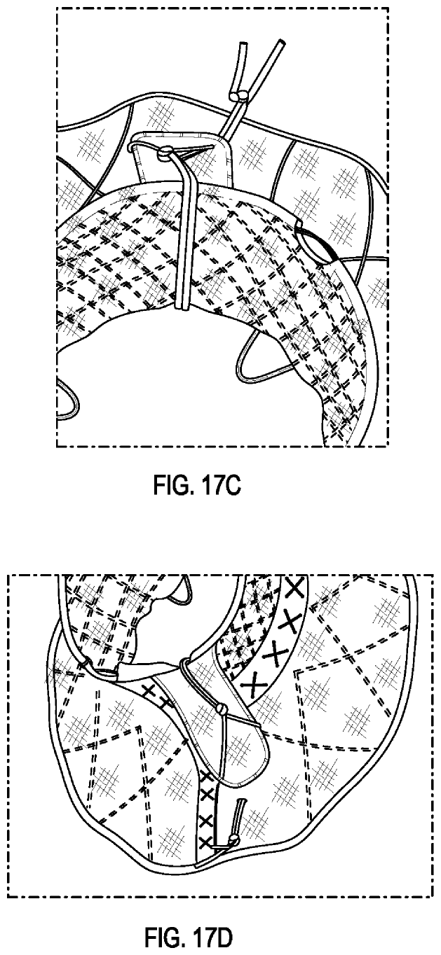

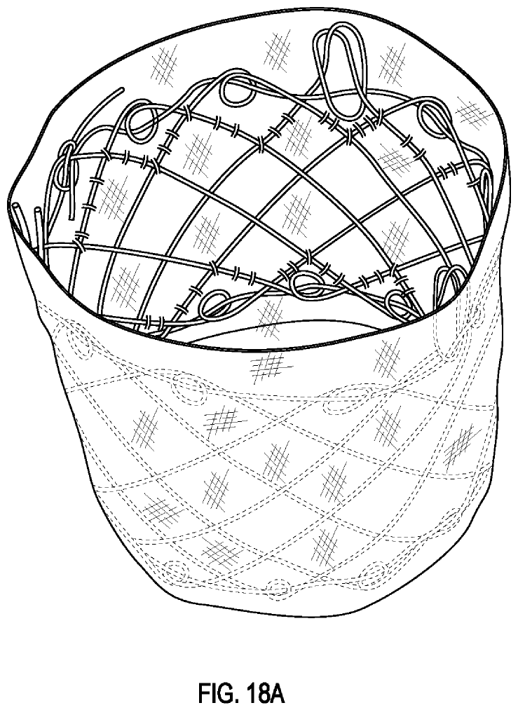

FIGS. 17A-17D generally illustrate embodiments of a valve replacement as disclosed herein. FIGS. 17A-17D show the attachment configurations for collapsible anchors and clips and further disclose a close-up view of a suture pattern that is used to collapse and control the anchors from all angles of the Valve Replacement. In these embodiments, a delivery component, such as one comprising one or more suture lines, is connected on a first to the engagement attachment, wherein the one or more suture lines connects on a second end to a controlling mechanism.

Valve Assembly

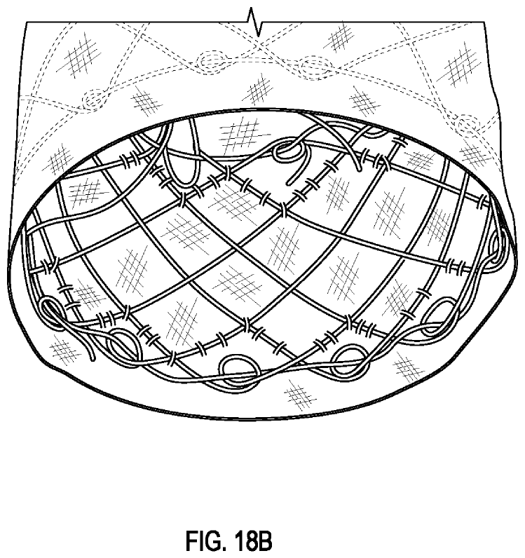

FIGS. 18A and 18B generally illustrate embodiments of a valve replacement as disclosed herein. As shown in FIGS. 18A and 18B, an embodiment of the Valve Replacement may be fabricated using a constraint to hold an Adapter frame at a specific dimension while attaching material to influence device performance. A fabrication technique is disclosed, which acts to influence the disposition of a braided wire frame—removing the inherent freedom of movement and unpredictability that is present between relative members of the frame structure when in a load-free state. This technique involves restraining the radial expansion of the frame with a constraint, such as feeding some number of sutures through or around the structure to hold it at a specific dimension other than its unrestrained, “free” dimension. In subsequent fabrication steps, the structure is incorporated into an assembly that adopts this new configuration and considers this to be the final dimension. When the constraints are removed from the braided frame, this braided frame tries to recover to its original “free” dimension—applying additional radial force to the surrounding structure while being constrained to the desired dimension.

The degree of radial force transmitted to the fabric material from the frame can be adjusted as required to achieve the optimal combination or performance properties. In particular, the strain energy density of the structure can be more uniform. A greater stiffness is achieved (resulting in a better seal) with less material, resulting in a more low-profile structure. The suture finally provides a biasing of the structure toward a desirable diameter and height for the valve structure.

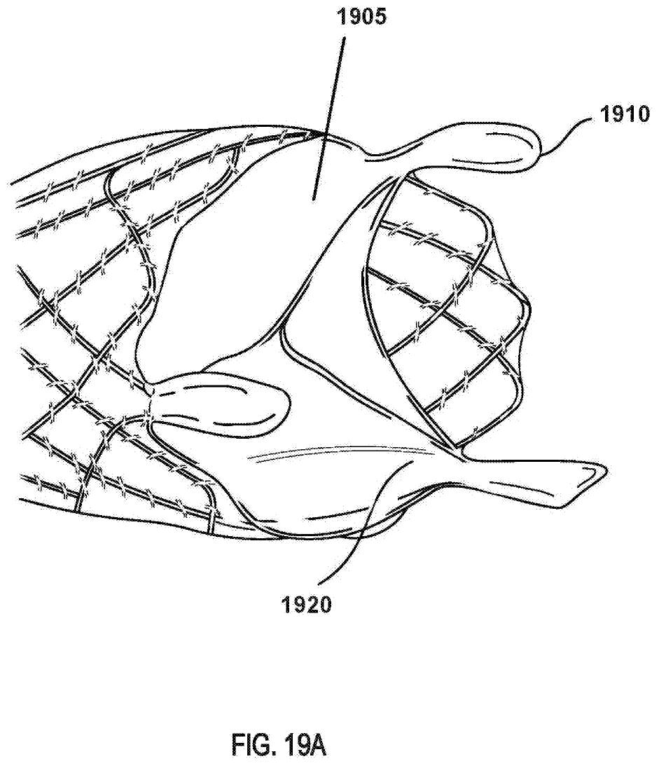

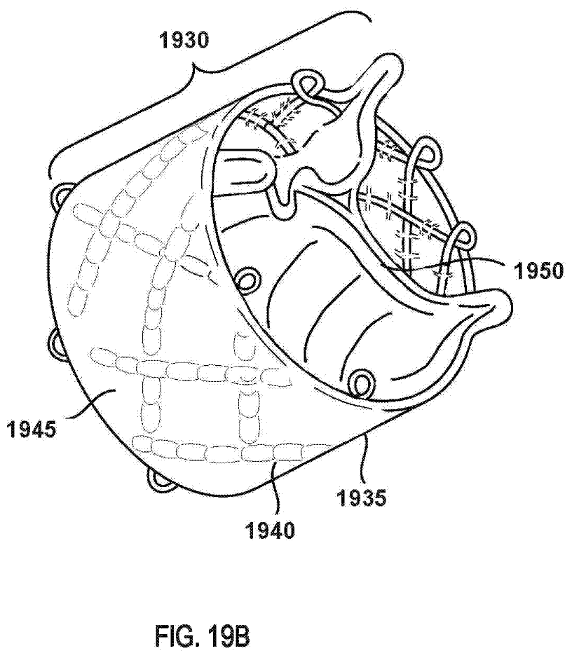

FIGS. 19A and 19B generally illustrate embodiments of a valve replacement as disclosed herein. FIG. 19A generally illustrates an embodiment of a Valve Assembly wherein leaflets 1905 are assembled to each other and/or to the frame by sewing. The leaflets are joined at commissure seams 1910 and then sewn, welded or otherwise attached to the commissure posts as well as to other points on the frame such as wires or wire intersections, or to materials attached to the frame, for example the leaflets being attached to a cuff, which cuff is then attached to the frame. Once the assembly is complete, the leaflets work in concert to close on the outflow (distal side when being implanted into leaflets a mitral valve) when fluid pressure is increased distally, so that the leaflets close or co-apt work in a Y pattern 1920.

As shown in FIG. 19B, a Valve Assembly 1930 comprises a braided frame a cuff covering 1935 on the outside. This cuff over the complete outer frame may serve as an extended sealing zone. A belly stitch 1940 may be sewn to the frame whereas a bellows stitch 1945 is not sewn to the frame. In this embodiment, the distal leaflet ends 1950 are shown coapting so as to close the valve in a loose Y-shape. In some embodiments, the valve co-apt area may comprise some “looseness” so as to ensure sufficient and effective contact among all three leaflets and ensure complete closing of the valve. Leaflets may be constructed of tissue such as porcine pericardium or other materials known to the art. In some cases, valves or parts of valves excised from animals may be sewn into the disclosed frame structure.

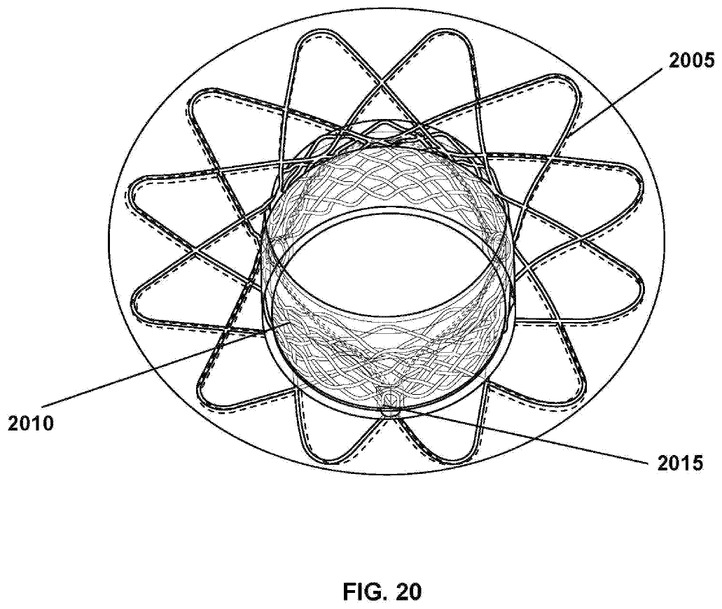

FIG. 20 generally illustrates an embodiment of a valve replacement as disclosed herein. FIG. 20 shows a bottom perspective view of the One-Piece System comprising a braided wire frame making up a flange 2005, an adapter body 2010, tabs 2015 compatible with leaflets, and leaflets 2020.

Other embodiments may include combinations and sub-combinations of features described or shown in the several figures, including for example, embodiments that are equivalent to providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, “feature” or “features” can refer to structures and/or functions of an apparatus, article of manufacture or system, and/or the steps, acts, or modalities of a method.

References throughout this specification to “one embodiment,” “an embodiment,” “an example embodiment,” etc., indicate that the embodiment described may include a particular feature, structure, or characteristic, but every embodiment may not necessarily include that particular feature, structure, or characteristic. Moreover, such phrases are not necessarily referring to the same embodiment. Further, when a particular feature, structure, or characteristic is described in connection with one embodiment, it will be within the knowledge of one skilled in the art to affect such feature, structure, or characteristic in connection with other embodiments whether or not explicitly described.

Unless the context clearly indicates otherwise (1) the word “and” indicates the conjunctive; (2) the word “or” indicates the disjunctive; (3) when the article is phrased in the disjunctive, followed by the words “or both,” both the conjunctive and disjunctive are intended; and (4) the word “and” or “or” between the last two items in a series applies to the entire series.

Where a group is expressed using the term “one or more” followed by a plural noun, any further use of that noun to refer to one or more members of the group shall indicate both the singular and the plural form of the noun. For example, a group expressed as having “one or more members” followed by a reference to “the members” of the group shall mean “the member” if there is only one member of the group.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

What is claimed:

1. A device for assisting the functioning of a heart valve, comprising:
a tubular braided frame comprising an inflow end and an outflow end;
a flange structure at the inflow end of the tubular braided frame;
at least one anchor at the outflow end of the tubular braided frame;
at least one commissure tab at the outflow end of the tubular braided frame; and
a leaflet assembly connected to the at least one commissure tab, wherein the leaflet assembly is configured to provide a seal between the inflow end and the outflow end of the tubular braided frame.

2. The device of claim 1, wherein the tubular braided frame further comprises at least one braided wire wound in a helical spiral direction, wherein the helical spiral direction begins at the inflow end and ends at the outflow end; and wherein the tubular braided frame is configured to lengthen and compress in relation to a heart contraction.

3. The device of claim 2, wherein the at least one braided wire comprises a first material type of wire, wherein the first material type of wire comprises a first wire type, a first bundle of wires, a first strip, a first rod, a first tube or a combination thereof.

4. The device of claim 3, wherein one or both of the flange structure and the at least one anchor comprises one or both of the first wire type and a second material type of wire, wherein the second material type of wire comprises a second wire type, a second bundle of wires, a second strip, a second rod, a second tube or a combination thereof.

5. The device of claim 2, wherein one or both of the flange structure and the at least anchor is configured to be radially overlapping with the tubular braided frame.

6. The device of claim 2, wherein a layer of material extends over one or more of the following: an outside portion of the tubular braided frame, an inside portion of the tubular braided frame, a top portion of the flange structure, a bottom portion of the flange structure, a top portion of the at least one anchor, and a bottom portion of the at least one anchor.

7. The device of claim 2, wherein the tubular braided frame further comprises an engagement attachment, wherein the engagement attachment comprises a barb, a hook, a clip, or combinations thereof.

8. The device of claim 6, wherein the layer of material extending over the outside portion of the tubular braided frame comprises one or more belt loops at one or both of the inflow end and the outflow end of the tubular braided frame.

9. The device of claim 2, wherein the tubular braided frame comprises a radial force when in a compressed state; wherein the tubular braided frame is configured to expand in relation to the radial force when the device is delivered to the heart valve.

10. A device for assisting the functioning of a heart valve, comprising:
an adapter comprising a tubular braided adapter frame that comprises an inflow end and an outflow end;
a flange structure at the inflow end of the tubular braided adapter frame; and
at least one anchor at the outflow end of the tubular braided adapter frame.

11. The device of claim 10, further comprising:
a valve assembly;
wherein the valve assembly comprises a tubular braided valve-assembly frame comprising a second inflow end and a second outflow end;
wherein the valve assembly comprises at least one commissure tab at the second inflow end;
wherein the valve assembly comprises a leaflet assembly connected to the at least one commissure tab;
wherein the leaflet assembly is configured to provide a seal between the second inflow end and the second outflow end.

12. The device of claim 11, wherein the valve assembly is configured to removably engage with the adapter, wherein the inflow end of the adapter is proximal in location to the second inflow end and the outflow end of the adapter is proximal in location to the second outflow end.

13. The device of claim 10, wherein the tubular braided adapter frame further comprises at least one braided wire wound in a helical spiral direction, wherein the helical spiral direction begins at the inflow end and ends at the outflow end; and wherein the at least one braided wire wound in a helical spiral direction is configured to lengthen and compress in relation to a heart contraction.

14. The device of claim 11, wherein the tubular braided valve-assembly frame further comprises at least one braided wire wound in a helical spiral direction, wherein the helical spiral direction begins at the second inflow end and ends at the second outflow end; and wherein the at least one braided wire wound in a helical spiral direction is configured to lengthen and compress in relation to a heart contraction.

15. The device of claim 13, wherein the at least one braided wire comprises a first material type of wire, wherein the first material type of wire comprises a first wire type, a first bundle of wires, a first strip, a first rod, a first tube or a combination thereof.

16. The device of claim 15, wherein one or both of the flange structure and the at least one anchor comprises one or both of the first wire type and a second material type of wire, wherein the second material type of wire comprises a second wire type, a second bundle of wires, a second strip, a second rod, a second tube or a combination thereof.

17. The device of claim 10, wherein one or both of the flange structure and the at least anchor is configured to be radially overlapping with the tubular braided adapter frame.

18. The device of claim 10, wherein a layer of material extends over one or more of the following: an outside portion of the tubular braided adapter frame, an inside portion of the tubular braided adapter frame, a top portion of the flange structure, a bottom portion of the flange structure, a top portion of the at least one anchor, and a bottom portion of the at least one anchor.

19. The device of claim 10, wherein the tubular braided frame further comprises an engagement attachment, wherein the engagement attachment comprises a barb, a hook, a clip, or combinations thereof.

20. The device of claim 18, wherein the layer of material extending over the outside portion of the tubular braided adapter frame comprises one or more belt loops at one or both of the inflow end and the outflow end of the tubular braided adapter frame.

21. The device of claim 14, wherein one or both of the tubular braided adapter frame and the tubular braided valve-assembly frame comprises a radial force when in a compressed state and is configured to expand in relation to the radial force when delivered to the heart valve.

22. A device for assisting the functioning of a heart valve, comprising:
A valve assembly comprising a tubular braided frame;
wherein the valve assembly comprises an inflow end and an outflow end;
wherein the valve assembly comprises at least one commissure tab at the inflow end; and
wherein the valve assembly comprises a leaflet assembly connected to the at least one commissure tab, wherein the leaflet assembly is configured to provide a seal between the inflow end and the outflow end.

23. The device of claim 22, wherein the tubular braided frame comprises at least one braided wire wound in a helical spiral direction, wherein the helical spiral direction begins at the inflow end and ends at the outflow end; and wherein the valve assembly is configured to lengthen and compress in relation to a heart contraction.

24. The device of claim 23, wherein the valve assembly comprises a first material type of wire, wherein the first material type of wire comprises a first wire type, a first bundle of wires, a first strip, a first rod, a first tube or a combination thereof.

25. The device of claim 22, wherein a layer of material extends over one or both of an outside portion of the valve assembly and an inside portion of the valve assembly.

26. The device of claim 23, wherein the tubular braided frame comprises a radial force when in a compressed state; wherein the tubular braided frame is configured to expand in relation to the radial force when the device is delivered to the heart valve.

27. A device for assisting the functioning of a heart valve, comprising:
a tubular frame comprising an inflow end and an outflow end;
wherein the tubular frame comprises at least one braided wire wound in a helical spiral direction;
wherein the helical spiral direction begins at the inflow end and ends at the outflow end;
wherein the tubular frame is configured to lengthen and compress in relation to a heart contraction.

28. The device of claim 27, wherein the tubular frame comprises a first material type of wire, wherein the first material type of wire comprises a first wire type, a first bundle of wires, a first strip, a first rod, a first tube or a combination thereof.

29. The device of claim 27, wherein a layer of material extends over one or both of an outside portion of the tubular frame and an inside portion of the tubular frame.

30. The device of claim 27, wherein the tubular frame comprises a radial force when in a compressed state; wherein the tubular frame is configured to expand in relation to the radial force when the device is delivered to the heart valve.
